(19) United States
(12) Patent Application Publication
(10) Pub. No.: US 2023/0354819 A1
(43) Pub. Date: Nov. 9, 2023

(54) Materials and Methods for Improving Plant Health

(71) Applicants: BASF SE, Ludwigshafen am Rhein (DE); RWTH AACHEN, Aachen (DE)

(72) Inventors: Tobias MAY, Ludwigshafen (DE); Evangeline Priya HAAS, Ludwigshafen (DE); Daniel Christoph HEINRICH, Ludwigshafen (DE); Jennifer GOLDMANNS, Aachen (DE); Jochen BUECHS, Aachen (DE)

(21) Appl. No.: 18/040,362

(22) PCT Filed: Jul. 30, 2021
(86) PCT No.: PCT/EP2021/071388

§ 371 Date: Feb. 2, 2023

(30) Foreign Application Priority Data
Aug. 3, 2020 (EP) 20189146.2

(51) Int. Cl. A01N 63/28; A01N 63/25; A01N 63/22; A01P 3/00; C12N 1/20

(52) U.S. Cl. CPC A01N 63/28; A01N 63/25; A01N 63/22; A01P 3/00; C12N 1/20

(57) ABSTRACT

Described herein is a fermentation medium for production of a plant health promoting microorganism. The fermentation medium includes nicotinic acid and biotin. A concentration of nicotinic acid in the fermentation medium is at least 0.1 mg/l, and a concentration of biotin in the fermentation medium is at least 0.01 mg/l. The fermentation medium also includes methionine. A concentration of methionine in the fermentation medium is at least 0.01 g/l.

The present invention is concerned with the field of improving the health of plants and/or increasing the yield of plants. In particular, the invention is concerned with preventing, limiting or reducing a phytopathogenic fungal disease.

In view of these goals, the invention provides materials and methods of using such materials to achieve or promote any of the aforementioned goals. The invention also provides materials and methods for producing such plant health compositions.

### BACKGROUND OF THE INVENTION

In the field of controlling phytopathogenic fungi it is known to use biopesticides. Biopesticides are frequently preferred over traditional synthetic fungicides because they are usually considered less toxic, more specific to the target pest, faster decomposable in natural environments and can reduce the use of conventional pesticides, particularly in integrated pest management programs. In the context of the present invention, two classes of biopesticides are of particular interest: biochemical pesticides, which are naturally occurring substances, and microbial pesticides, in which a microorganism is an active ingredient. The present invention particularly intends to facilitate production of such biopesticides and to improve the efficacy thereof.

Compared to conventional pesticides, biopesticides are generally more expensive to produce and thus are economically disadvantaged, despite their ecological advantages. For the control of phytopathogenic fungi typically spore forming bacteria have been cultivated. For example, Ryu et al. (Applied Biochemistry and Biotechnology 2019) describe media and methods for cultivating Paenibacillus strains for the production of fusaricidins. Likewise, WO2018183383 describes media and cultivation methods for Paenibacillus strains for fusaricidin production. In both publications the cultivation media are either adapted to individual bacterial strains and thus cannot be generalised to other bacilli, or they rely on expensive media components, notably yeast extract, which leads to a price increase of correspondingly produced biocontrol agents such that they can hardly compete with traditionally synthesised fungicides.

The invention aspires to reduce or overcome the aforementioned disadvantages of the prior art while providing a plant health promotion composition which is microorganism-based or derived from microorganisms. In particular, the invention aspires to provide cultivation media (1) that are useful for a variety of biocontrol microorganisms and (2) which reduce the need for discouraged amino acid sources, for example yeast extract, and/or (3) which notably increase the production of antifungal agents by said microorganisms. In this regard, the invention also aspires to provide cultivation methods based on the fermentation media of the present invention.

### SUMMARY OF THE INVENTION

The invention correspondingly provides a fermentation medium for the production of a plant health promoting microorganism, preferably an antifungal microorganism, comprising
nicotinic acid and biotin,
wherein the concentration of nicotinic acid in the fermentation medium is at least 0.1 mg/l, preferably at least 2 mg/l, more preferably at least 5 mg/l, more preferably 5-100 mg/l and more preferably 10-100 mg/l, and wherein the concentration of biotin in the fermentation medium is at least 0.01 mg/l, preferably at least 0.05 mg/l, more preferably 0.05-1000 mg/l, more preferably at least 0.12 mg/l, more preferably 0.12-1000 mg/l,
and
methionine,
wherein the concentration of methionine in the fermentation medium is at least 0.01 g/l, preferably at least 0.1 g/l, more preferably at least 0.2 g/l, more preferably 0.2-3 g/l.

The invention furthermore provides a fermentation method comprising the step of cultivating a microorganism culture comprising or consisting of one or more plant health promoting microorganisms,
wherein the contents of a fermentation medium according to the present invention is provided to the culture within a period of at most 72 h.

The invention also provides a plant health promotion composition obtainable or obtained by a fermentation method according to the present invention.

Furthermore, the invention provides a plant material, preferably a plant propagation material, comprising on its surface a composition according to the present invention.

The invention also provides a use of a plant health promotion composition of the present invention for preventing, limiting or reducing a phytopathogenic fungal disease and/or for improving the health of a plant and/or for increasing yield of plants.

And the invention provides a method for preventing, limiting or reducing a phytopathogenic fungal disease and/or for increasing the health of a plant, comprising applying an effective amount of the composition according to the invention to the plant, a part or propagation material thereof or to the soil where the plants are to grow.

### BRIEF DESCRIPTION OF THE FIGURES

FIG. 1 shows the dependence of optical density (OD) on medium composition. Increase of nicotinic acid alone accounts for most of the increase in microorganism biomass, as evidenced by OD.

FIG. 2 shows the development of final OD depending on nicotinic acid concentration in the cultivation of example 2. With increasing nicotinic acid concentration the maximal bacterial growth is achieved.

FIG. 3 shows the concentration of total fusaricidins A, B and D in the fermentation broth obtained in example 2. The fusaricidin concentration is increased at higher initial concentrations of nicotinic acid. At approximately 10 mg/l nicotinic acid the increase in fusaricidin concentration starts to level off.

FIG. 4 shows the final OD depending on biotin concentration. In the absence of biotin the time for reaching maximum bacterial growth speed is markedly delayed and final biomass concentration is reduced.

FIG. 5 shows the concentration of fusaricidins in the fermentation broth after 48 h of fermentation. The fusaricidin concentration is highest in a medium with lowest initial yeast extract content and high initial concentration of DL-methionine and does not increase further at elevated concentrations of yeast.

FIG. 6 shows the development of optical density depending on medium composition. Bacterial growth speed, as indicated by OD increase, is maintained in those media which comprise a reduced content of yeast extract and increased concentration of DL-methionine and nicotinic acid compared to a full yeast extract medium.

FIG. 7 shows the development of fusaricidin concentration relative to maximum fusaricidin concentration for a yeast extract containing medium depending on medium composition. Fusaricidin concentration is higher in those media, which comprise a reduced content of yeast extract and increased concentration of DL-methionine and nicotinic acid.

FIG. 8 shows the development of OD depending on the presence of salts. Maximal bacterial growth, as indicted by optical density, is prolonged in the presence of salts.

FIG. 9 shows the development of fusaricidin concentration depending on the presence of salts. Fusaricidin concentrations increase faster and reach a higher maximum in the presence of salts compared to a corresponding medium without addition of salts.

FIG. 10 shows the development of oxygen transfer rate (OTR) depending on medium composition. Maximal bacterial metabolic activity, as indicted by OTR, is highest for a medium of the present invention compared to prior art media when normalising for sugar source concentration.

FIG. 11 shows the development of fusaricidin concentration (total of fusaricidins A, B and D) depending on medium composition. Fusaricidin concentration is highest for a medium of the present invention compared to prior art media when normalising for sugar source concentration.

FIG. 12 shows the efficacy of cell-free culture filtrate against Fusarium graminearum after cultivation of various plant health promoting microorganisms in different media. Same concentration of carbon source was used in each approach. Generally, efficacy is improved for filtrates obtained after cultivation of those organisms in media according to the present invention.

FIG. 13 shows the efficacy of cell-free culture filtrate against Botrytis cinerea after cultivation of Paenibacillus. Generally, efficacy is improved for filtrates obtained after cultivation in media according to the present invention.

FIG. 14 shows the normalized concentration of total fusaricidins A, B and D obtained for 18 Paenibacillus polymyxa chemical NTG mutants compared to the wild type progenitor in different media. After NTG chemical mutagenesis fusaricidin production was significantly increased in a minimal medium according to the present invention compared to a complex medium in 15 out of 18 mutants tested. Furthermore, for 7 mutants fusaricidin production was clearly higher in the minimal medium compared to fusaricidin production of the wild type strain in complex medium. Only 4 mutants showed a clearly improved fusaricidin production in complex medium.

FIG. 15 shows the total fusaricidin A, B and D yield for the same strains and media as depicted in FIG. 14. Yield is approximated by dividing total fusaricidin A, B and D concentration by OD600. For all mutants fusaricidin yield is increased, compared to the wild type, in minimal medium according to the present invention. Only one mutant shows improved fusaricidin yield compared to the wild type when cultivated in complex medium.

### DETAILED DESCRIPTION OF THE INVENTION

The present invention provides a fermentation medium. According to the present invention, a fermentation medium is a solid, semisolid or preferably a liquid to sustain or grow microorganisms. The fermentation medium of the present invention preferably is suitable for cultivation of microorganisms in a bioreactor tank. According to the present invention, the terms “growth”, “cultivation” and “fermentation” of microorganisms are used interchangeably and denote a process in which one or more microorganisms are contacted with, preferably immersed in, a fermentation medium sustaining their metabolism such that nutrients acquired from the fermentation medium allow the microorganisms to multiply and optionally also to sporulate.

The fermentation medium of the present invention is suitable and designed for the production of a plant health promoting microorganism. A plant health promoting microorganism can act as a microbial biopesticide. The microorganism for cultivation in the fermentation medium of the present invention preferably is a prokaryotic microorganism. Suitable and particularly preferred microorganisms are described below. The microorganisms promote plant health by inhibiting the growth of plant pathogens, for example by feeding on such pathogens or preventing their maturation or generation of offspring, particularly of fungal spores. In addition, or alternatively, the microorganisms may also produce metabolites inhibiting one or more plant pathogens. For example, several Paenibacillus microorganisms produce fusaricidins which are enriched in or attached to the spores. When the spores are applied to a plant tissue, fusaricidins present in the product or fermentation broth directly inhibit or kill fungal plant pathogens and the germinated and growing bacterial cells secure further long-term supply of such antifungal compounds.

The fermentation medium of the present invention is preferably suitable for or adapted to the cultivation of an antifungal microorganism as described herein. The term fungus according to the present invention is to be understood widely and denotes any microorganism responsible for the development of mildews, rots, wilts, blights or spots on plants. In particular, according to the invention, the term fungus denotes a microorganism responsible for any of the fungal diseases described herein. Fungi of particular concern to the present invention are also described herein. Correspondingly, the term antifungal microorganism or antifungal compound denotes a microorganism or a component or substance capable of preventing, limiting or reducing one or more fungal induced plant diseases as described herein.

The fermentation medium of the present invention comprises nicotinic acid, biotin and methionine. It has been surprisingly found that these components not only promote the growth of plant health promoting microorganisms, in particular of genus Paenibacillus, but also increase the antifungal activity of microorganisms fermented in a medium of the present invention compared to standard microbial media. Furthermore, the components of the fermentation medium of the present invention surprisingly allow to reduce the content of discouraged media components, notably yeast extract, without reducing the antifungal activity of the microorganism grown in the fermentation medium of the present invention.

Unless denoted otherwise herein, the concentrations of components of the fermentation medium of the present invention are calculated on the basis of the respective substances as such. For example, when a fermentation medium of the present invention contains methionine in the form of a salt or ester thereof, the concentration must be increased accordingly to make up for the additional salt or ester component.

Furthermore, unless explicitly declared to the contrary concentrations of fermentation media components refer, per unit volume of fermentation medium, to the amount of added components and thus not necessarily to the total amount of said components per unit volume. Where a fermentation medium of the present invention comprises complex media components, in particular a discouraged component, and notably yeast extract or yeast autolysate, the concentrations resulting from the presence of such substances in the complex media components are disregarded. For example, when a fermentation medium of the present invention comprises 10 g/l yeast extract, then the components of the fermentation medium of the present invention are nevertheless added in the respective amount per unit volume, for example 5-100 mg/l nicotinic acid, 0.05-1000 mg/l biotin and 0.2-3 g/l methionine are preferably added.

As described herein, the addition of nicotinic acid not only promotes the growth of plant health promoting microorganisms, for example of genus Paenibacillus, but also advantageously leads to an increase in fusaricidin yield. This was very surprising in view of the aforementioned publication by Ryu et al. 2019 who had already described a fermentation medium purportedly optimised for fusaricidin production.

It has furthermore been surprisingly found that the addition of biotin is not essential for the growth of plant health promoting microorganisms, for example of genus Paenibacillus, but advantageously promotes growth of such microorganisms. In combination, the addition of nicotinic acid and biotin allows for a fast generation of plant health promoting microorganism biomass and, as applicable, of corresponding spores. In essence, the period required for the production of a plant health promotion composition of the present invention is thus advantageously shortened. This, in turn, increases the output per year of a fermentation plant for the production of plant health promotion compositions of the present invention, thereby improving cost effectiveness of the plant health promotion composition of the present invention.

It has also surprisingly been found that methionine is an essential component for reducing the content of discouraged media components, particularly yeast extract or yeast autolysate, without compromising the yield of antifungal compounds produced by a plant health promoting microorganism fermented in a fermentation medium of the present invention. It was particularly surprising that even after reduction of yeast extract content by 85%, the concentration of fusaricidin at the end of the fermentation had even increased without the fermentation taking longer. In this regard, the addition of methionine like the addition of nicotinic acid and biotin favourably allows to reduce the time needed for fermentation of a set quantity of antifungal compounds, particularly of fusaricidin, and/or increases the output of such components when the fermentation time is not reduced.

The concentration of nicotinic acid in the fermentation medium of the present invention is at least 0.1 mg/l, preferably at least 2 mg/l, more preferably at least 5 mg/l, and more preferably 5-100 mg/l. As described above, this is to say that at least 0.1 mg, preferably at least 2 mg, more preferably at least 5 mg, more preferably 5-100 mg and more preferably 10-100 mg is added to one litre of fermentation medium.

The concentration of biotin in the fermentation medium of the present invention is at least 0.01 mg/l, preferably at least 0.05 mg/l, more preferably 0.05-1000 mg/l, more preferably at least 0.12 mg/l, more preferably 0.12-1000 mg/l. Also as described above, this is to say that at least 0.01 mg, preferably at least 0.05 mg, more preferably 0.05-1000 mg, more preferably at least 0.12 mg, more preferably 0.12-1000 mg is added to one litre of fermentation medium.

The concentration of methionine in the fermentation medium of the present invention is at least 0.01 g/l, preferably at least 0.1 g/l, more preferably at least 0.2 g/l, more preferably 0.2-3 g/l. As described above, this is to say at least 0.01 g, preferably at least 0.1 g, more preferably at least 0.2 g, more preferably 0.2-3 g is added to one litre of fermentation medium.

The fermentation medium according to the present invention preferably further comprises a slow release amino acid source. It has surprisingly been found that by providing a slow release amino acid source instead of a supply of individual amino acids in comparable concentrations can increase the speed of antifungal substance synthesis. In particular, replacing a slow release amino acid source like soy meal by an equivalent amount of free amino acids can delay reaching the final concentration of fusaricidin produced by Paenibacillus by a factor of two or three.

The slow release amino acid source is selected from one or more protein sources, one or more protein hybrid sources and, less preferred, one or more discouraged sources. Those slow release amino acid source can also comprise a mixture of one or more protein sources and one or more protein, hydrolysate sources, one or more protein sources and one or more discouraged sources, one or more protein hydrolysate sources and one or more discouraged sources, and a combination of one or more protein sources, one or more protein hydrolysate sources and one or more discouraged sources.

The protein sources of the slow release amino acid source according to the invention are selected from the group consisting of corn steep liquor, milk protein, skim milk protein, whey protein, casein, pea protein, cotton seed protein, wheat gluten protein, porcine protein, bovine protein, gelatine, egg protein, fish protein, microbial protein, soy protein and soy meal. Preferred protein sources are corn steep liquor, pea protein, cotton seed protein, microbial protein and soy meal, more preferred protein sources are corn steep liquor, soy protein and soy meal, most preferred protein source is soy meal. For the purposes of the present invention, the protein source can be in any form, for example, low-fat or defatted soy meal or low-fat soy flour and toasted or untoasted soy meal.

The protein hydrolysate sources of those slow release amino acid source according to the invention are selected from the group consisting of hydrolysates of one or more of the aforementioned protein sources, tryptose (peptone from protein mixture, tryptic digest), proteose-peptone, peptone from animal protein, casein hydrolysate, peptone from casein, tryptone (peptone from casein), peptone from gelatine, lactalbumin hydrolysate, liver hydrolysate, peptone from meat, peptone from porcine heart, peptone from plant protein, peptone from broadbean, gluten hydrolysate from maize, peptone from pea, peptone from potatoes, peptone from soybean, peptone from soybean meal, peptone from wheat, peptone from fungal protein and potato infusion powder. Preferred protein hydrolysate sources are peptone from soybean, peptone from soybean meal, peptone from wheat and peptone from pea.

In a slow release amino acid source for a fermentation medium of the present invention, the following sources are discouraged: brain extract, in particular from porcine brain; brain heart infusion; heart extract, in particular from bovine heart; heart infusion powder, in particular from bovine heart; meat extract; yeast autolysate and yeast extract. These sources are discouraged because they are very complex and thus vary in composition for different charges of the discouraged sources, particularly in the case of yeast autolysate and yeast extract. These sources generally are more expensive than the aforementioned protein or protein hydrolysate sources. Thus, even though it may be necessary that the fermentation medium according to the present invention for particular fermentation purposes comprises one or more of the discouraged sources, nevertheless, the invention provides ways of reducing the content of such discouraged sources without compromising product quality, for example, the fermentation speed of a plant health promoting microorganism or fusaricidin production.

When present the total concentration of the aforementioned slow release amino acid source in the fermentation medium according to the present invention is 0-100 g/l, preferably 0.1-100 g/l. In the light of the examples presented herein, the skilled person is capable of selecting a concentration suitable for his particular fermentative need.

The invention in particular provides a fermentation medium, wherein the total concentration of yeast extract and yeast autolysate in the fermentation medium comprising a slow release amino acid source is 0-8 g/l, preferably 0-3 g/l, and wherein particularly preferred the total concentration of discouraged sources in the fermentation medium is 0-8 g/l, preferably 0-3 g/l. As shown in the examples herein, the fermentation medium according to the present invention allows to decrease the concentration of discouraged slow release amino acid sources and in particular of yeast extract without compromising the speed of growth of plant health promoting microorganisms, in particular of genus Paenibacillus, while simultaneously, and, surprisingly, also allowing to more than double the production of fusaricidins, even compared to purportedly optimised media for fusaricidin production.

The fermentation medium according to the present invention preferably further comprises a sugar source. The sugar source serves as a carbon source in the fermentation of the plant health promoting microorganism, and also as an energy source. The sugar source according to the fermentation medium is selected from the group consisting of glucose, dextrose, starch, fructose, galactose, xylose, xylitol, inulin, sorbitol, fucose, molasses, sucrose, lactose, glycerol, pectin, galacturonic acid, maltose, maltodextrin, maltotriose and higher maltooligosacharides or maltose syrup or mixtures thereof. For the purposes of describing the present invention, the concentration of maltose syrup is calculated on the basis of a 50% by weight aqueous syrup; when the maltose concentration in the syrup is less than 50%, the volume of maltose syrup has to be adjusted accordingly. When present the total concentration of the aforementioned sugar source in the medium is at least 5 g/l, preferably at least 40 g/l, more preferably 50-400 g/l.

Further preferably, the fermentation medium according to the present invention further comprises
MnS_{O4}*H₂O: 1-1000 mg/l, preferably 8-100 mg/l
CuSO₄*5H₂O: 0.1-100 mg/l, preferably 2-8 mg/l
Na₂MoO₄*2H₂O: 0.1-50 mg/l, preferably 1-5 mg/l
Fe₂(SO₄)₃*H₂O: 0.8-1000 mg/l, preferably 5-50 mg/l
citric acid: 0.1-100 g/l, preferably 0.5-20 g/l
and optionally
Ca(NO₃)₂*4H₂O: 0-3 g/l, preferably 0-1 g/l.

It has been surprisingly found that the addition of such substances leads to a further increase in the production of antifungal substances by plant health promoting microorganisms. For example, it has been surprisingly found that the speed of production and final concentration of fusaricidin in fermentations using microorganisms of genus Paenibacillus are increased. Furthermore, it has been surprisingly found that in a variety of plant health promoting microorganisms the antifungal activity of a cell free material harvested from such fermentation is increased.

Further preferably according to the present invention, the fermentation medium further comprises
one or more or, preferably, all of the following amino acids
Histidine: at least 10 mg/l, preferably 50-1000 mg/l,
Prolin: at least 10 mg/l, preferably 300-1000 mg/l,
Arginin: at least 10 mg/l, preferably 50-1000 mg/l,
Glutamate: at least 10 mg/l, preferably 200-5000 mg/l,
and optionally one or more or, preferably, all of the following amino acid
Cystein: at least 10 mg/l, preferably 50-1000 mg/l, most preferably 300-600 mg/l
Tryptophan: at least 10 mg/l, preferably 50-1000 mg/l, most preferably 200-500 mg/l.

The addition of the aforementioned amino acids allows to reduce the content of the slow release amino acid source, preferably of soy flour, without reducing the achievable total fusaricidin concentration.

The invention in particular provides a preferred fermentation medium, wherein
the concentration of discouraged slow release amino acid source in the fermentation medium is 0-3 g/l, and the concentration of yeast extract and yeast autolysate in the fermentation medium is 0-3 g/l,
the concentration of the total sugar source in the fermentation medium is 10-100 g/l and the sugar source preferably comprises or consists of maltose, maltodextrin, maltotriose and higher maltooligosacharides or maltose syrup, and
the concentration of the total slow release amino acid protein or protein hydrolysate source is 5-100 g/l, and the slow release amino acid source preferably comprises or consists of soy meal or a hydrolysate thereof.

Such fermentation media allow to achieve the advantages conveyed by the present invention. Such fermentation media are further described in the examples below.

The invention also provides a fermentation method. In the fermentation method of the present invention, a microorganism culture is cultivated. The term “cultivation” denotes any microbiological process to increase, over a cultivation time, the amount of desired components by feeding one or more microorganisms for the production of such components with suitable nutrients in a suitable environment. For the purposes of the present invention, the suitable nutrients are provided by the fermentation medium of the present invention or are added to a fermentation medium as described herein. The desired components to be produced according to the present invention can be the microorganisms themselves, spores or cysts thereof or metabolites, for example fusaricidins, produced by the microorganisms during fermentation. The microorganisms according to the present invention are plant health promoting microorganisms; preferred microorganisms are described herein.

The fermentation method can be performed as a batch method wherein microorganisms are provided in a fermenter comprising a fermentation medium, the microorganisms are then cultivated in the fermenter and finally the fermenter contents are harvested. The fermentation may also be performed as or comprise a fed batch step wherein during fermentation additional components are added to the fermentation medium, thereby increasing the volume of fermenter contents until the nominal fermenter volume is reached. This can be done for example by continuously feeding additional components to the fermentation medium or by providing intermittent bolus feeds. It is also possible for a batch or fed batch fermentation that during harvesting the fermenter contents are not completely removed, but some volume is left as an inoculant in the fermenter for the next fermentation batch. Both processes can be combined. The fermentation method of the present invention can also be performed as a continuous culture, for example as a turbidostat or chemostat.

In the fermentation method of the present invention, the contents of a fermentation medium of the present invention is provided to the microorganism. This can be achieved by providing a fermentation medium of the present invention, adding a starter microorganism culture comprising one or more plant health promoting microorganisms to the medium and cultivating the culture. Another way of conducting the fermentation is to provide a medium, adding a starter microorganism culture comprising one or more plant health promoting microorganisms to the medium and, within cultivation, adding the contents of a fermentation medium of the present invention. Addition of the contents of a fermentation medium can be performed in one step, continuously or repeatedly during cultivation. When a plant health promoting microorganism used in such fermentation is capable of spore production it is advisable to prevent starvation for sufficient time in order to delay sporulation and to increase number of vegetative cells until entry into sporulation to achieve high final spore concentrations.

When the contents of the fermentation medium of the present invention are added during cultivation, the following effective dosages relative to the fermentation broth volume are preferred:
nicotinic acid: at least 0.04 mg/(l d), preferably at least 0.86 mg/(l d), more preferably at least 2 mg/(l d), more preferably 2-66 mg/(l d), biotin: at least 0.004 mg/(l d), preferably at least 0.02 mg/(l d), more preferably 0.02-660 mg/(l d), more preferably at least 0.05 mg/(l d), more preferably 0.05-660 mg/(l d),
methionine: at least 4 mg/(l d), preferably at least 40 mg/(l d), more preferably at least 80 mg/(l d), more preferably 0.08-2 g/(l d).

The aforementioned effective dosage for a fermentation medium component are calculated by summing the mass of the component added during a period of 24 h ending at an endpoint chosen at will and dividing by the fermentation broth volume during the period of 24 h. For example, assuming a fermentation to which a component is added at 2 h, 4 h, 6 h and 28 h after fermentation start (t=0). Then the fermentation method of the present invention can be performed if, for each obligatory component of the fermentation medium of the present invention, the total amount of the respective component present in the fermentation medium at t=0 and after addition at 2 h, 4 h and/or 6 h and divided by the fermentation broth volume at 24 h falls within the above definition of concentrations or effective dosages for a fermentation medium according to the present invention. However, the fermentation method of the present invention can also be performed if, for each obligatory component of the fermentation medium of the present invention, the total amount of the respective component present in the fermentation medium at 4 h, 6 h and 28 h divided by the fermentation broth volume at 28 h falls within the above definition of concentrations or effective dosages for a fermentation medium according to the present invention. The fermentation method of the present invention thus advantageously allows for a flexible dosage regime.

It is preferred but not necessary that all obligatory and/or facultative components of the fermentation medium of the present invention are added in constant amounts. Instead, it is also advantageous to first allow a rapid growth of the cultivated one or more microorganisms by adding a sugar source initially in a high amount and reducing the amount added in later additions. Furthermore, it is preferred to increase the amount of slow release amino acid sources in additions after 24 h and reduce the amount of slow release amino acid sources in additions at and after onset of sporulation.

The preferred effective dosages of further independent components of the fermentation medium of the present invention are:
the total of slow release amino acid sources as defined in claim 2: 0-100 g/(l d), preferably 0.04-100 g/(l d), wherein preferably the total concentration of yeast extract and yeast autolysate in the fermentation medium during cultivation is 0-5 g/(l d), preferably 0-1.3 g/(l d), and wherein particularly preferred the total concentration of discouraged sources in the fermentation medium during cultivation is 0-5 g/(l d), preferably 0-1.3 g/(l d),
total of sugar sources as defined in claim 4 preferably is at least 2 g/(l d), preferably at least 17 g/(l d)), more preferably 12-270 g/(l d),
MnSO₄*H₂O: preferably 1-670 mg/(l d), more preferably 3-67 mg/(l d)
SCuSO₄*5H₂O: preferably 0.04-67 mg/(l d), more preferably 0.86-5.5 mg/(l d)
Na₂MoO₄*2H₂O: preferably 0.1-10 mg/(l d), more preferably 1-5 mg/(l d)
Fe₂(SO₄)₃*H₂O: preferably 0.3-670 mg/(l d), more preferably 2-35 mg/(l d)
citric acid: preferably 0.04-67 g/(l d), more preferably 0.2-15 g/(l d)
Ca(NO₃)₂*4H₂O: preferably 0-2 g/(l d), more preferably 0-0.5 g/(l d)
preferably one or more or, more preferably, all of the following amino acids
Histidine: at least 4 mg/(l d), preferably 21-670 mg/(l d),
Prolin: at least 4 mg/(l d), preferably 120-670 mg/(l d),
Arginin: at least 4 mg/(l d), preferably 21-670 mg/(l d),
Glutamate: at least 4 mg/(l d), preferably 85-670 mg/(l d),
preferably one or more or, more preferably, all of the following amino acids:
Cysteine: at least 4 mg/(l d), preferably 21-670 mg/(l d), most preferably 120-400 mg/(l d)
Tryptophan: at least 4 mg/(l d), preferably 21-670 mg/(l d), most preferably 85-350 mg/(l d)

With these effective dosages the advantages described above with respect to the individual substances or substance groups can be achieved.

The fermentation method of the present invention is preferably performed with a fermentation medium, wherein the components thereof are present in a concentration higher than the respective minimum concentration except for the discouraged slow release amino acid source. Thus, for example, the initial fermentation medium comprises at least 2 mg/l nicotinic acid, at least 0.05 mg/l biotin, at least 0.1 g/l methionine, and preferably also comprises at least 20 g/l maltose syrup (50% by weight), and also preferably comprises at least 3 g/l, more preferably at least 6 g/l soy meal. Likewise, where components of the fermentation medium of the present invention are added over the cultivation period, it is preferred that the amounts added are higher than the minimal amounts for a fermentation medium of the present invention, except for the discouraged slow amino acid release source.

The fermentation medium of the present invention preferably is for a microorganism used in the fermentation method of the present invention such that the microorganism culture preferably comprises or consists of one or more biocontrol microorganisms selected from the group consisting of the taxonomic ranks:
phylum Firmicutes, more preferably of class Bacilli, more preferably of order Bacillales, more preferably of any of:
family Bacillaceae, more preferably of genus Bacillus;
family Paenibacillaceae, more preferably of genus Paenibacillus;
phylum Proteobacteria, more preferably of class Gammaproteobacteria, more preferably of order Pseudomonadales, more preferably of family Pseudomonadaceae, more preferably of genus Pseudomonas;
phylum Proteobacteria, more preferably of class Betaproteobacteria, more preferably of order Burkholderiales, more preferably of family Burkholderiaceae, more preferably of any of: genus Burkholderia;
genus Paraburkholderia;
phylum Proteobacteria, more preferably of class Alphaproteobacteria, more preferably of order Rhizobiales, more preferably of any of:
family Rhizobiaceae, more preferably of genus Rhizobium;
family Bradyrhizobiaceae, more preferably of genus Bradyrhizobium;
family Rhizobiaceae, more preferably of genus Sinorhizobium;
phylum Proteobacteria, more preferably of class Alphaproteobacteria, more preferably of order Sphingomonadales, more preferably of family Sphingomonadaceae, more preferably of genus Sphingomonas;
phylum Actinobacteria, more preferably of class Actinobacteria, more preferably of order Streptomycetales, more preferably of family Streptomycetaceae, more preferably of genus Streptomyces;
phylum Bacteroidetes, more preferably of class Flavobacteria, more preferably of order Flavobacteriales, more preferably of family Flavobacteriaceae, more preferably of genus Chryseobacterium;
phylum Actinobacteria, more preferably of class Actinobacteria, more preferably of order Corynebacteriales, more preferably of family Nocardiaceae, more preferably of genus Rhodococcus;

Members of these taxonomic ranks are known for their plant health promoting activity and preferably because of their antifungal activity. As shown in the examples, a fermentation medium of the present invention used in a fermentation method of the present invention is useful to generate products of the present invention effective against a variety of fungal plant diseases. In particular members of the following species are preferred for inclusion or formation of the microorganism culture in the fermentation method of the present invention:
Paenibacillus species: P. abekawaensis, P. abyssi, P. aceris, P. aceti, P. aestuarii, P. agarexedens, P. agaridevorans, P. alba, P. albidus, P. albus, P. alginolyticus, P. algorifonticola, P. alkaliterrae, P. alvei, P. amylolyticus, P. anaericanus, P. antarcticus, P. antibioticophila, P. antri, P. apiaries, P. apiarius, P. apis, P. aquistagni, P. arachidis, P. arcticus, P. assamensis, P. aurantiacus, P. azoreducens, P. azotifigens, P. baekrokdamisoli, P. barcinonensis, P. barengoltzii, P. beijingensis, P. borealis, P. bouchesdurhonensis, P. bovis, P. brasilensis, P. brassicae, P. bryophyllum, P. caespitis, P. camelliae, P. camerounensis, P. campinasensis, P. castaneae, P. catalpae, P. cathormii, P. cavernae, P. cellulosilyticus, P. cellulositrophicus, P. chartarius, P. chibensis, P. chinensis, P. chinjuensis, P. chitinolyticus, P. chondroitinus, P. chungangensis, P. cineris, P. cisolokensis, P. contaminans, P. cookii, P. crassostreae, P. cucumis, P. curdlanolyticus, P. daejeonensis, P. dakarensis, P. darangshiensis, P. darwinianus, P. dauci, P. dendritiformis, P. dongdonensis, P. donghaensis, P. doosanensis, P. durus, P. edaphicus, P. ehimensis, P. elgii, P. elymi, P. endophyticus, P. enshidis, P. esterisolvens, P. etheri, P. eucommiae, P. faecis, P. favisporus, P. ferrarius, P. filicis, P. flagellatus, P. fonticola, P. forsythiae, P. frigoriresistens, P. fujiensis, P. fukuinensis, P. gansuensis, P. gelatinilyticus, P. ginsengagri, P. ginsengarvi, P. ginsengihumi, P. ginsengiterrae, P. glacialis, P. glebae, P. glucanolyticus, P. glycanilyticus, P. gorillae, P. graminis, P. granivorans, P. guangzhouensis, P. harenae, P. helianthi, P. hemerocallicola, P. herberti, P. hispanicus, P. hodogayensis, P. hordei, P. horti, P. humicus, P. hunanensis, P. ihbetae, P. ihuae, P. ihumii, P. illinoisensis, P. insulae, P. intestini, P. jamilae, P. jilunlii, P. kobensis, P. koleovorans, P. konkukensis, P. konsidensis, P. koreensis, P. kribbensis, P. kyungheensis, P. lactis, P. lacus, P. larvae, P. lautus, P. lemnae, P. lentimorbus, P. lentus, P. liaoningensis, P. limicola, P. lupini, P. luteus, P. lutimineralis, P. macerans, P. macquariensis, P. marchantiophytorum, P. marinisediminis, P. marinum, P. massiliensis, P. maysiensis, P. medicaginis, P. mendelii, P. mesophilus, P. methanolicus, P. mobilis, P. montanisoli, P. montaniterrae, P. motobuensis, P. mucilaginosus, P. nanensis, P. naphthalenovorans, P. nasutitermitis, P. nebraskensis, P. nematophilus, P. nicotianae, P. nuruki, P. oceanisediminis, P. odorifer, P. oenotherae, P. oralis, P. oryzae, P. oryzisoli, P. ottowii, P. ourofinensis, P. pabuli, P. paeoniae, P. panacihumi, P. panacisoli, P. panaciterrae, P. paridis, P. pasadenensis, P. pectinilyticus, P. peoriae, P. periandrae, P. phocaensis, P. phoenicis, P. phyllosphaerae, P. physcomitrellae, P. pini, P. pinihumi, P. pinisoli, P. pinistramenti, P. pocheonensis, P. polymyxa, P. polysaccharolyticus, P. popilliae, P. populi, P. profundus, P. prosopidis, P. protaetiae, P. provencensis, P. psychroresistens, P. pueri, P. puernese, P. puldeungensis, P. purispatii, P. qingshengii, P. qinlingensis, P. quercus, P. radicis, P. relictisesami, P. residui, P. rhizoplanae, P. rhizoryzae, P. rhizosphaerae, P. rigui, P. ripae, P. rubinfantis, P. ruminocola, P. sabinae, P. sacheonensis, P. salinicaeni, P. sanguinis, P. sediminis, P. segetis, P. selenii, P. selenitireducens, P. senegalensis, P. senegalimassiliensis, P. seodonensis, P. septentrionalis, P. sepulcri, P. shenyangensis, P. shirakamiensis, P. shunpengii, P. siamensis, P. silagei, P. silvae, P. sinopodophylli, P. solanacearum, P. solani, P. soli, P. sonchi group, P. sophorae, P. spiritus, P. sputi, P. stellifer, P. susongensis, P. swuensis, P. taichungensis, P. taihuensis, P. taiwanensis, P. taohuashanense, P. tarimensis, P. telluris, P. tepidiphilus, P. terrae, P. terreus, P. terrigena, P. tezpurensis, P. thailandensis, P. thermoaerophilus, P. thermophilus, P. thiaminolyticus, P. tianmuensis, P. tibetensis, P. timonensis, P. translucens, P. tritici, P. triticisoli, P. tuaregi, P. tumbae, P. tundrae, P. turicensis, P. tylopili, P. typhae, P. tyrfis, P. uliginis, P. urinalis, P. validus, P. velaei, P. vini, P. vortex, P. vorticalis, P. vulneris, P. wenxiniae, P. whitsoniae, P. wooponensis, P. woosongensis, P. wulumuqiensis, P. wynnii, P. xanthanilyticus, P. xanthinilyticus, P. xerothermodurans, P. xinjiangensis, P. xylanexedens, P. xylaniclasticus, P. xylanilyticus, P. xylanisolvens, P. yanchengensis, P. yonginensis, P. yunnanensis, P. zanthoxyli, P. zeae,
preferably P. agarexedens, P. agaridevorans, P. alginolyticus, P. alkaliterrae, P. alvei, P. amylolyticus, P. anaericanus, P. antarcticus, P. assamensis, P. azoreducens, P. barcinonensis, P. borealis, P. brassicae, P. campinasensis, P. chinjuensis, P. chitinolyticus, P. chondroitinus, P. cineris, P. curdlanolyticus, P. daejeonensis, P. dendritiformis, P. ehimensis, P. elgii, P. favisporus, P. glucanolyticus, P. glycanilyticus, P. graminis, P. granivorans, P. hodogayensis, P. illinoisensis, P. jamilae, P. kobensis, P. koleovorans, P. koreensis, P. kribbensis, P. lactis, P. larvae, P. lautus, P. lentimorbus, P. macerans, P. macquariensis, P. massiliensis, P. mendelii, P. motobuensis, P. naphthalenovorans, P. nematophilus, P. odorifer, P. pabuli, P. peoriae, P. phoenicis, P. phyllosphaerae, P. polymyxa, P. popilliae, P. rhizosphaerae, P. sanguinis, P. stellifer, P. taichungensis, P. terrae, P. thiaminolyticus, P. timonensis, P. tylopili, P. turicensis, P. validus, P. vortex, P. vulneris, P. wynnii, P. xylanilyticus,
particularly preferred Paenibacillus koreensis, Paenibacillus rhizosphaerae, Paenibacillus polymyxa, Paenibacillus amylolyticus, Paenibacillus terrae, Paenibacillus polymyxa polymyxa, Paenibacillus polymyxa plantarum, Paenibacillus nov. spec epiphyticus, Paenibacillus terrae, Paenibacillus macerans, Paenibacillus alvei, more preferred Paenibacillus polymyxa, Paenibacillus polymyxa polymyxa, Paenibacillus polymyxa plantarum, Paenibacillus nov. spec epiphyticus, Paenibacillus terrae, Paenibacillus macerans, Paenibacillus alvei, even more preferred Paenibacillus polymyxa, Paenibacillus polymyxa polymyxa, Paenibacillus polymyxa plantarum and Paenibacillus terrae.
Bacillus species: B. abyssalis, B. acanthi, B. acidiceler, B. acidicola, B. acidiproducens, B. aciditolerans, B. acidopullulyticus, B. acidovorans, B. aeolius, B. aequororis, B. aeris, B. aerius, B. aerolacticus, B. aestuarii, B. aidingensis, B. akibai, B. alcaliinulinus, B. alcalophilus, B. algicola, B. alkalicola, B. alkalilacus, B. alkalinitrilicus, B. alkalisediminis, B. alkalitelluris, B. alkalitolerans, B. alkalogaya, B. altitudinis, B. alveayuensis, B. amiliensis, B. andreesenii, B. andreraoultii, B. aporrhoeus, B. aquimaris, B. arbutinivorans, B. aryabhattai, B. asahii, B. aurantiacus, B. australimaris, B. azotoformans, B. bacterium, B. badius, B. baekryungensis, B. bataviensis, B. benzoevorans, B. beringensis, B. berkeleyi, B. beveridgei, B. bingmayongensis, B. bogoriensis, B. borbori, B. boroniphilus, B. butanolivorans, B. cabrialesii, B. caccae, B. camelliae, B. campisalis, B. canaveralius, B. capparidis, B. carboniphilus, B. casamancensis, B. caseinilyticus, B. catenulatus, B. cavernae, B. cecembensis, B. cellulosilyticus, B. chagannorensis, B. chandigarhensis, B. cheonanensis, B. chungangensis, B. ciccensis, B. cihuensis, B. circulans, B. clausii, B. coagulans, B. coahuilensis, B. cohnii, B. composti, B. coniferum, B. coreaensis, B. crassostreae, B. crescens, B. cucumis, B. dakarensis, B. daliensis, B. danangensis, B. daqingensis, B. decisifrondis, B. decolorationis, B. depressus, B. deramificans, B. deserti, B. dielmoensis, B. djibelorensis, B. drentensis, B. ectoiniformans, B. eiseniae, B. enclensis, B. endolithicus, B. endophyticus, B. endoradicis, B. endozanthoxylicus, B. farraginis, B. fastidiosus, B. fengqiuensis, B. fermenti, B. ferrariarum, B. filamentosus, B. firmis, B. firmus, B. flavocaldarius, B. flexus, B. foraminis, B. fordii, B. formosensis, B. fortis, B. freudenreichii, B. fucosivorans, B. fumarioli, B. funiculus, B. galactosidilyticus, B. galliciensis, B. gibsonii, B. ginsenggisoli, B. ginsengihumi, B. ginsengisoli, B. glennii, B. glycinifermentans, B. gobiensis, B. gossypii, B. gottheilii, B. graminis, B. granadensis, B. hackensackii, B. haikouensis, B. halmapalus, B. halodurans, B. halosaccharovorans, B. haynesii, B. hemicellulosilyticus, B. hemicentroti, B. herbersteinensis, B. hisashii, B. horikoshii, B. horneckiae, B. horti, B. huizhouensis, B. humi, B. hunanensis, B. hwajinpoensis, B. idriensis, B. indicus, B. infantis, B. infernus, B. intermedius, B. intestinalis, B. iocasae, B. isabeliae, B. israeli, B. jeddahensis, B. jeotgali, B. kexueae, B. kiskunsagensis, B. kochii, B. kokeshiiformis, B. koreensis, B. korlensis, B. kribbensis, B. krulwichiae, B. kwashiorkori, B. kyonggiensis, B. lacisalsi, B. lacus, B. lehensis, B. lentus, B. ligniniphilus, B. lindianensis, B. litoralis, B. loiseleuriae, B. lonarensis, B. longiquaesitum, B. longisporus, B. luciferensis, B. luteolus, B. luteus, B. lycopersici, B. magaterium, B. malikii, B. mangrovensis, B. mangrovi, B. mannanilyticus, B. manusensis, B. marasmi, B. marcorestinctum, B. marinisedimentorum, B. marisflavi, B. maritimus, B. marmarensis, B. massiliglaciei, B. massilioanorexius, B. massiliogabonensis, B. massiliogorillae, B. massilionigeriensis, B. massiliosenegalensis, B. mediterraneensis, B. megaterium, B. mesonae, B. mesophilum, B. mesophilus, B. methanolicus, B. miscanthi, B. muralis, B. murimartini, B. nakamurai, B. nanhaiisediminis, B. natronophilus, B. ndiopicus, B. nealsonii, B. nematocida, B. niabensis, B. niacini, B. niameyensis, B. nitritophilus, B. notoginsengisoli, B. novalis, B. obstructivus, B. oceani, B. oceanisediminis, B. ohbensis, B. okhensis, B. okuhidensis, B. oleivorans, B. oleronius, B. olivae, B. onubensis, B. oryzae, B. oryzaecorticis, B. oryzisoli, B. oryziterrae, B. oshimensis, B. pakistanensis, B. panacisoli, B. panaciterrae, B. paraflexus, B. patagoniensis, B. persicus, B. pervagus, B. phocaeensis, B. pichinotyi, B. piscicola, B. piscis, B. plakortidis, B. pocheonensis, B. polygoni, B. polymachus, B. populi, B. praedii, B. pseudalcaliphilus, B. pseudofirmus, B. pseudoflexus, B. pseudomegaterium, B. psychrosaccharolyticus, B. pumilus, B. purgationiresistens, B. qingshengii, B. racemilacticus, B. rhizosphaerae, B. rigiliprofundi, B. rubiinfantis, B. ruris, B. safensis, B. saganii, B. salacetis, B. salarius, B. salidurans, B. salis, B. salitolerans, B. salmalaya, B. salsus, B. sediminis, B. selenatarsenatis, B. senegalensis, B. seohaeanensis, B. shacheensis, B. shackletonii, B. shandongensis, B. shivajii, B. similis, B. simplex, B. sinesaloumensis, B. siralis, B. smithii, B. solani, B. soli, B. solimangrovi, B. solisilvae, B. songklensis, B. spongiae, B. sporothermodurans, B. stamsii, B. subterraneus, B. swezeyi, B. taeanensis, B. taiwanensis, B. tamaricis, B. taxi, B. terrae, B. testis, B. thaonhiensis, B. thermoalkalophilus, B. thermoamyloliquefaciens, B. thermoamylovorans, B. thermocopriae, B. thermolactis, B. thermophilus, B. thermoproteolyticus, B. thermoterrestris, B. thermozeamaize, B. thioparans, B. tianmuensis, B. tianshenii, B. timonensis, B. tipchiralis, B. trypoxylicola, B. tuaregi, B. urumqiensis, B. vietnamensis, B. vini, B. vireti, B. viscosus, B. vitellinus, B. wakoensis, B. weihaiensis, B. wudalianchiensis, B. wuyishanensis, B. xiamenensis, B. xiaoxiensis, B. zanthoxyli, B. zeae, B. zhangzhouensis, B. zhanjiangensis, preferably Bacillus licheniformis, B. megaterium, B. subtilis, B. pumilus, B. firmus, B. thuringiensis, B. velezensis, B. linens, B. atrophaeus, B. amyloliquefaciens, B. aryabhattai, B. cereus, B. aquatilis, B. circulans, B. clausii, B. sphaericus, B. thiaminolyticus, B. mojavensis, B. vallismortis, B. coagulans, B. sonorensis, B. halodurans, B. pocheonensis, B. gibsonii, B. acidiceler, B. flexus, B. hunanensis, B. pseudomycoides, B. simplex, B. safensis, B. mycoides,
particularly preferred B. amyloliquefaciens, B. thuringiensis, B. velezensis, B. subtilis and B. megatherium.
Pseudomonas species: P. aureofaciens, P. cepacia, P. corrugata, P. fluorescens, P. putida, P. aeruginosa, P. chlororaphis, P. koreensis (lurida), P. nitroreducens, P. syringae, P. lini, P. indica, P. mandelii (subgroup), P. rhodesiae, P. rhizosphaere, P. psychrotolerans, P. abietaniphila, P. extremorientalis, P. lutea, P. pictorum, P. deceptionensis, P. cissicola, preferably P. fluorescens, P. syringae and P. putida,
Burkholderia species: B. phytofirmans, B. gladioli, B. cepacia, B. anthina, B. arboris, B. seminalis, B. ambifaria, B. caledonica, B. cenocepacia, B. contaminans, B. dolosa, B. glumae, B. graminis, B. kururiensis, B. multivorans, B. pyrrocinia, B. sacchari, B. silvatlantica, B. stabilis, B. tropica, B. unamae, B. vietnamiensis, B. xenovorans, B. caribensis, B. mimosarum, B. nodosa, B. phymatum, B. tuberum, B. calva, B. kirkii, B. nigropunctata, B. glumae, B. caryophylii, B. fungorum, B. megapolitana, B. ginsengisoli, B. terricola, B. phenoliruptrix, B. sedimincola, B. bryophila, B. phenazinium,
preferably B. cepacia and B. contaminans
Paraburkholderia species: P. caledonica, P. phytofirmans, P. terricola, P. terrae P. hospital, P. jirisanensis, P. caribensis, P. tropica, P. megapolitana
preferably P. phytofirmans
Rhizobiales: Rhizobium qilianshanense, R. mayense, R. miluonense, R. tropici, R. lusitanum, R. viscosum, R. lusitanum, R. metallidurans, R. mayense, R. phaseoli, R. tropici, R. multihospitium, R. japonicum, R. leguminosarum, R. leguminosarum bv. phaseoli, R. leguminosarum bv. viciae, R. leguminosarum bv. trifolii, R. trifolii, R. lupine, Bradyrhizobium japonicum, Sinorhizobium meliloti, Mesorihizobium meliloti, Ensifer meliloti,
preferably Bradyrhizobium japonicum, Rhizobium japonicum, R. leguminosarum and R. leguminosarum bv. phaseoli,
Sphingomonas species: S. yabuuchiae, S. oligoaromativorans, S. azotifigens, S. trueperi, S. azotifigens, S. yabuuchiae, S. trueperi, S. kyeonggiensis, S. trueperi, S. taxi, S. panni, S. endophytica, S. panni, S. panacis, S. pituitosa, S. aerolata, S. parapaucimobilis,
preferably S. parapaucimobilis and S. sanguinis
Streptomyces species: S. araujoniae, S. barakatei, S. beta-vulgaris, S. hygroscopicus, S. rimosus, S. anulatus, S. halstedii, S. tsusimaensis, S. setonii, S. albospinus, S. lydicus, S. kurssanvii, S. griseorubens, S. miharaensis, S. corchorusii, S. mutabilis, S. roseodiastaticus, S. erumpens, S. aurantiacus, S. rameus, S. psammoticus, S. thermocarboxydus, S. bikiniensis, S. goshikiensis, S. spectabilis, S. roseochromogenus, S. fulvoviolaceus, S. rochei, S. griseiviridis, S. asterosporus, S. chibaensis, S. antibioticus, S. peruviensis, S. kasugaensis, S. griseus var. autotrophicus, S. olivaceus, S. flavofuscus, S. luteogriseus, S. spororaveus, S. alboflavus, S. tendae, S. griseoruginosus, S. violaceusniger, S. griseus, S. plicatus, S. chattanoogensis, S. natalensis, S. gilvosporeus, S. pseudovenezuelae, S. wadayamensis, S. thioluteus, S. griseoviridis and S. eurocidicus,
preferably S. platensis, S. flavogriseus, S. lavendulae, S. hygroscopicus and S. lydicus Chryseobacterium species: C. aquaticum, C. hagamense, C. indologenes, C. taeanense, C. nepalense, C. lathyri, C. zeae, C. taiwanense, C. aquifrigidense, C. soldanellicola, C. balustinum,
preferably C. indologenes and C. soldanellicola
Rhodococcus species: R. erythropolis, R. rhodochrous, R. fascians and R. equi preferably R. erythropolis and R. fascians
Virgibacillus species: V. marismortui, Terribacillus halophilus, Halomonas elongate, Lanococcus rifietoensis, Staphylococcus equorum, Erwinia herbicola, Pantoea agglomerans, Glucanobacter cerinus, Lactobacillus plantarum, Pediococcus acidilactici, Pediococcus pentosaceus, Serratia plymuthica, Brevibacillus brevis,
preferably Brevibacillus brevis and Lactobacillus plantarum.

As shown in the examples particularly good results have been obtained with such microorganisms.

Preferred plant health promoting microorganisms belong to the genera Paenibacillus or Bacillus as described above, with microorganisms of genus Paenibacillus being even more preferred. Most preferred plant health promoting microorganisms are Paenibacillus polymyxa, Paenibacillus polymyxa plantarum and Paenibacillus terrae.

The microorganism culture in the fermentation method of the present invention preferably is a mixed culture consisting of different species of microorganisms and/or different strains of a species of microorganisms. The invention thus provides a fermentation method useful for cultivating consortia of plant health producing microorganisms.

Alternatively, the microorganism culture in the fermentation method of the present invention preferably is a pure culture consisting of one species of one microorganism and even more preferably consists of one strain of one species of a microorganism. The fermentation method of the present invention in this embodiment is particularly easy to control using standard microbiological and biotechnological techniques.

When at least one microorganism of the microorganism culture during cultivation in the fermentation method of the present invention produces spores, then preferably such spores are harvested. Harvesting techniques like centrifugation, filtration and gear filtration are known to the person skilled in the art. It is a particular advantage of the fermentation method of the present invention that high titers of spores with a high content of antifungal substances, notably fusaricidins, can be produced with low efforts in a conveniently short time and with high antifungal activity.

It is also preferred to harvest a cell free suspension at the end of the fermentation method of the present invention. Again, techniques for obtaining a cell free suspension unknown to the person skilled in the art can favourably be combined with methods for harvesting spores.

The invention also provides a plant health promotion composition, obtainable or obtained by a method according to the present invention. As described herein, such compositions are surprisingly effective, and they are easy and fast and cost effectively to produce.

The plant health composition optionally further comprises a stabiliser, preferably as disclosed in WO2019222253A, and also preferably one or more fusaricidins. Fusaricidins are a group of antibiotics isolated from Paenibacillus spp. from the class of cyclic lipodepsipeptides which often share the following structural features: a macrocyclic ring consisting of 6 amino acid residues, three of which are L-Thr, D-allo-Thr and D-Ala, as well as the 15-guanidino-3-hydroxypentadecanoic acid tail attached to the N-terminal L-Thr residue by an amide bond (ChemMedChem 7, 871-882, 2012; J. Microbiol. Meth. 85, 175-182, 2011). These compounds are cyclized by a lactone bridge between the N-terminal L-Thr hydroxyl group and the C-terminal D-Ala carbonyl group. The position of the amino acid residues within the depsipeptide cycle are usually numbered starting with the abovementioned L-Thr which itself also carries the GHPD chain and ending with the C-terminal D-Ala. Non-limiting examples of fusaricidins isolated from Paenibacillus are designated LI-F03, LI-F04, LI-F05, LI-F07 and LI-F08 (J. Antibiotics 40(11), 1506-1514, 1987; Heterocycles 53(7), 1533-1549, 2000; Peptides 32, 1917-1923, 2011) and fusaricidins A (also called LI-F04a), B (also called LI-F04b), C (also called LI-F03a) and D (also called LI-F03b) (J. Antibiotics 49(2), 129-135, 1996; J. Antibiotics 50(3), 220-228, 1997). The amino acid chain of a fusaricidin is not ribosomally generated but is generated by a non-ribosomal peptide synthetase. Among isolated fusaricidin antibiotics, fusaricidin A has shown the most promising antimicrobial activity against a variety of clinically relevant fungi and gram-positive bacteria such a Staphylococcus aureus (MIC value range: 0.78-3.12 g/ml) (ChemMedChem 7, 871-882, 2012). Fusaricidins A, B, C and D are also reported to inhibit plant pathogenic fungi such as Fusarium oxysporum, Aspergillus niger, Aspergillus oryzae, and Penicillium thomii (J. Antibiotics 49(2), 129-135, 1996; J. Antibiotics 50(3), 220-228, 1997). Fusaricidins such as Li-F05, LI-F07 and LI-F08 have been found to have certain antifungal activity against various plant pathogenic fungi such as Fusarium moniliforme, F. oxysporum, F. roseum, Giberella fujkuroi, Helminthosporium sesamum and Penicillium expansum (J. Antibiotics 40(11), 1506-1514, 1987). Fusaricidins also have antibacterial activity to Gram-positive bacteria including Staphylococcus aureus (J. Antibiotics 49, 129-135, 1996; J. Antibiotics 50, 220-228, 1997).

In addition, fusaricidins have antifungal activity against Leptosphaeria maculans which causes black root rot of canola (Can. J. Microbiol. 48, 159-169, 2002). Moreover, fusaricidins A and B and two related compounds thereof produced by certain Paenibacillus strains were found to induce resistance reactions in cultured parsley cells and to inhibit growth of Fusarium oxysporum (WO 2006/016558; EP 1788074A1). In WO 2016/020371 it was found that the whole culture broth, the culture medium and cell-free extracts of the bacterial strains Lu16774, Lu17007 and Lu17015 show inhibitory activity inter alia against Alternaria spp., Botrytis cinerea and Phytophthora infestans.

Furthermore, the plant health composition of the present invention preferably further comprises
a) one or more microbial pesticides with fungicidal, bactericidal, viricidal and/or plant defense activator activity,
b) one or more biochemical pesticides with fungicidal, bactericidal, viricidal and/or plant defense activator activity,
c) one or more microbial pesticides with insecticidal, acaricidal, molluscidal and/or nematicidal activity,
d) one or more biochemical pesticides with insecticidal, acaricidal, molluscidal, pheromone and/or nematicidal activity,
e) one or more fungicide selected from respiration inhibitors, sterol biosynthesis inhibitors, nucleic acid synthesis inhibitors, inhibitors of cell division and cytoskeleton formation or function, inhibitors of amino acid and protein synthesis, signal transduction inhibitors, lipid and membrane synthesis inhibitors, inhibitors with multi-site action, cell wall synthesis inhibitors, plant defence inducers and fungicides with unknown mode of action.

The further components a)-d) are described in WO2017137353, which is incorporated herein for the purpose of enumerating the respective substances. The further components e) are described in WO2017137351, which is also incorporated herein for the purpose of enumerating the respective fungicides.

The invention also provides a plant material, preferably a plant propagation material, comprising on its surface a plant health composition according to the invention. Such application serves to materialise the advantageous plant health promotion properties of the composition of the present invention. The term “plant” is intended to encompass plants at any stage of maturity or development, as well as any tissues or organs (plant parts) taken or derived from any such plant unless otherwise clearly indicated by context. The term “plant material” denotes any tissue, organ or material produced by a plant, including, but are not limited to, plant cells, stems, roots, flowers, ovules, stamens, seeds, leaves, embryos, meristematic regions, callus tissue, anther cultures, gametophytes, sporophytes, pollen, microspores, protoplasts, hairy root cultures, straw, husks, fruit and nut shells. As used herein, a “plant cell” includes, but is not limited to, a protoplast, gamete producing cell, and a cell that regenerates into a whole plant. The term “plant propagation material” is to be understood to denote all the generative parts of the plant such as seeds and vegetative plant material such as cuttings and tubers (e.g. potatoes), which can be used for the multiplication of the plant. This includes seeds, roots, fruits, tubers, bulbs, rhizomes, shoots, sprouts and other parts of plants, including seedlings and young plants, which are to be transplanted after germination or after emergence from soil. These young plants may also be protected before transplantation by a total or partial treatment by immersion in or pouring of the plant health promotion composition of the present invention.

The plant health promotion composition of the present invention is preferably applied to the plant material, preferably a plant propagation material, by any step of dressing, spraying, coating, film coating, pelleting, dusting or soaking.

According to the invention the plant health promotion composition of the present invention is used for preventing, limiting or reducing a phytopathogenic fungal disease and/or for improving the health of a plant and/or for increasing plant yield. As described herein the composition of the present invention favourably improves plant health, preferably by preventing, limiting or reducing a phytopathogenic fungal disease when applied to a plant material, preferably the visible part of a plant and/or the roots thereof. Application of the composition can be performed on plants or parts thereof showing symptoms of a fungal disease to reduce the intensity or limit the spread of the disease. Application of the composition can also be performed on plants or parts thereof not showing symptoms of a fungal disease to prevent or delay the onset or spread of a disease. Treatment of plants or sufficient parts thereof results in an improved plant health and preferably in an increased yield.

It is a particular advantage that the present invention is useful for the preventing, limiting or reducing a phytopathogenic fungal disease, wherein
i) the fungal disease is selected from white blister, downy mildews, powdery mildews, clubroot, Sclerotinia rot, Fusarium wilts and rots, Botrytis rots, anthracnose, Rhizoctonia rots, damping-off, cavity spot, tuber diseases, rusts, black root rot, target spot, Aphanomyces root rot, Ascochyta collar rot, gummy stem blight, Alternaria leaf spot, black leg, ring spot, late blight, Cercospora, leaf blight, Septoria spot, leaf blight, or a combination thereof, and/or
ii) the fungal disease is caused or aggravated by a microorganism selected from the taxonomic ranks:
   class Sordariomycetes, more preferably of order Hypocreales, more preferably of family Nectriaceae, more preferably of genus Fusarium;
   class Sordariomycetes, more preferably of order Glomerellales, more preferably of family Glomerellaceae, more preferably of genus Colletotrichum;
   class Leotinomycetes, more preferably of order Helotiales, more preferably of family Sclerotiniaceae, more preferably of genus Botrytis;
   class Dothideomycetes, more preferably of order Pleosporales, more preferably of family Pleosporaceae, more preferably of genus Alternaria;
   class Dothideomycetes, more preferably of order Pleosporales, more preferably of family Phaeosphaeriaceae, more preferably of genus Phaeosphaeria;
   class Dothideomycetes, more preferably of order Botryosphaeriales, more preferably of family Botryosphaeriaceae, more preferably of genus Macrophomina;
   class Dothideomycetes, more preferably of order Capnodiales, more preferably of family Mycosphaerellaceae, more preferably of genus Zymoseptoria;
   class Agraricomycetes, more preferably of order Cantharellales, more preferably of family Ceratobasidiaceae, more preferably of genus Rhizoctonia or Thanatephorus;
   class Pucciniomycetes, more preferably of order Pucciniales, more preferably of family Pucciniaceae, more preferably of genus Uromyces or Puccinia;
   class Ustilaginomycetes, more preferably of order Ustilaginales, more preferably of family Ustilaginaceae, more preferably of genus Ustilago;
   class Oomycota, more preferably of order Pythiales, more preferably of family Pythiaceae, more preferably of genus Pythium;
   class Oomycota, more preferably of order Peronosporales, more preferably of family Peronosporaceae, more preferably of genus Phytophthora, Plasmopara or Pseudoperonospora.

Preferably the composition of the present invention is used to treat or useful against any of the following pathogens:
Fusarium species: F. acaciae-mearnsii, F. agapanthi, F. albidum, F. nematophilum, F. andiyazi, F. arthrosporioides, F. austroamericanum, F. azukicola, F. babinda, F. bambusae, F. boothii, F. brasilicum, F. buharicum, F. sublunatum, F. algeriense, F. beomiforme, F. burgessii, F. buxicola, F. cyanostomum, F. caeruleum, F. camptoceras, F. caucasicum, F. cavispermum, F. cerealis, F. atrovinosum, F. aywerte, F. chlamydosporum, F. humicola, F. microconidium, F. nelsonii, F. peruvianum, F. spinosum, F. sporodochiale, F. tjaynera, F. ciliatum, F. citricola, F. salinense, F. coeruleum, F. anguioides, F. austroafricanum, F. concolor, F. convolutans, F. cortaderiae, F. crassistipitatum, F. dactylidis, F. albosuccineum, F. decemcellulare, F. detonianum, F. biseptatum, F. delphinoides, F. dimerum, F. domesticum, F. lunatum, F. nectrioides, F. penzigii, F. diversisporum, F. ensiforme, F. eumartii, F. expansum, F. acutatum, F. ananatum, F. annulatum, F. anthophilum, F. awaxy, F. bactridioides, F. begoniae, F. brevicatenulatum, F. bulbicola, F. circinatum, F. coicis, F. concentricum, F. denticulatum, F. dlaminii, F. ficicrescens, F. fracticaudum, F. fractiflexum, F. fredkrugeri, F. fujikuroi, F. globosum, F. guttiforme, F. konzum, F. lactis, F. mangiferae, F. marasasianum, F. mexicanum, F. mundagurra, F. napiforme, F. nygamai, F. parvisorum, F. phyllophilum, F. pininemorale, F. proliferatum, F. pseudoanthophilum, F. pseudocircinatum, F. pseudonygamai, F. ramigenum, F. sacchari, F. secorum, F. sororula, F. subglutinans, F. succisae, F. temperatum, F. thapsinum, F. tjaetaba, F. udum, F. verticillioides, F. xylarioides, F. xyrophilum, F. fusarioides, F. gibbosum, F. heterosporum, F. hostae, F. hyaloacrosporum, F. aberrans, F. arcuatisporum, F. brevicaudatum, F. bubalinum, F. caatingaense, F. cateniforme, F. citri, F. clavum, F. coffeatum, F. compactum, F. croceum, F. duofalcatisporum, F. equiseti, F. fasciculatum, F. flagelliforme, F. gracilipes, F. guilinense, F. hainanense, F. humuli, F. incarnatum, F. ipomoeae, F. irregulare, F. lacertarum, F. longicaudatum, F. longifundum, F. luffae, F. monophialidicum, F. mucidum, F. multiceps, F. nanum, F. neosemitectum, F. pernambucanum, F. persicinum, F. scirpi, F. sulawesiense, F. tanahbumbuense, F. kyushuense, F. larvarum, F. lateritium, F. sarcochroum, F. stilboides, F. lunulosporum, F. macroceras, F. melanochlorum, F. meridionale, F. merismoides, F. mesoamericanum, F. musae, F. musarum, F. neocosmosporiellum, F. newnesense, F. commune, F. gaditjirri, F. lyarnte, F. miscanthi, F. nisikadoi, F. nurragi, F. oligoseptatum, F. ophiodes, F. callistephi, F. carminascens, F. contaminatum, F. cugenangense, F. curvatum, F. duoseptatum, F. elaeidis, F. fabacearum, F. foetens, F. glycines, F. gossypinum, F. hoodiae, F. inflexum, F. languescens, F. libertatis, F. nirenbergiae, F. odoratissimum, F. oxysporum, F. pharetrum, F. tardichlamydosporum, F. triseptatum, F. veterinarium, F. palustre, F. polyphialidicum, F. praegraminearum, F. pyriforme, F. redolens, F. reticulatum, F. riograndense, F. robustum, F. rusci, F. aethiopicum, F. armeniacum, F. asiaticum, F. brachygibbosum, F. culmorum, F. gerlachii, F. goolgardi, F. graminearum, F. langsethiae, F. longipes, F. louisianense, F. nepalense, F. nodosum, F. poae, F. pseudograminearum, F. sambucinum, F. sibiricum, F. sporotrichioides, F. transvaalense, F. venenatum, F. vorosii, F. sedimenticola, F. setosum, F. siculi, F. sinensis, F. ambrosium, F. brasiliense, F. cuneirostrum, F. euwallaceae, F. falciforme, F. floridanum, F. haematococcum, F. illudens, F. kelerajum, F. keratoplasticum, F. kuroshium, F. kurunegalense, F. lichenicola, F. mahasenii, F. obliquiseptatum, F. paranaense, F. petroliphilum, F. phaseoli, F. plagianthi, F. pseudensiforme, F. rectiphorum, F. rekanum, F. solani, F. striatum, F. tuaranense, F. tucumaniae, F. virguliforme, F. cicatricum, F. fuckelii, F. staphyleae, F. zealandicum, F. sterilihyphosum, F. subtropicale, F. sudanense, F. sulphureum, F. terricola, F. continuum, F. torreyae, F. zanthoxyli, F. torulosum, F. acuminatum, F. avenaceum, F. flocciferum, F. petersiae, F. tricinctum, F. tumidum, F. tupiense, F. ussurianum, F. ventricosum, F. verrucosum, F. zeae, most preferably Fusarium graminearum;
Colletotrichum species: C. acerbum, C. acidae, C. abscissum, C. acutatum, C. brisbanense, C. cairnsense, C. carthami, C. chrysanthemi, C. citri, C. cosmi, C. costaricense, C. cuscutae, C. fioriniae, C. godetiae, C. guajavae, C. indonesiense, C. javanense, C. laticiphilum, C. limetticola, C. lupini, C. melonis, C. nymphaeae, C. paranaense, C. paxtonii, C. salicis, C. scovillei, C. simmondsii, C. sloanei, C. tamarilloi, C. walleri, C. agaves, C. alcornii, C. ampelinum, C. arboricola, C. artocarpicola, C. arxii, C. atractylodicola, C. australe, C. axonopodi, C. baltimorense, C. annellatum, C. beeveri, C. boninense, C. brasiliense, C. brassicicola, C. constrictum, C. karsti, C. petchii, C. phyllanthi, C. brassicae, C. camelliae-japonicae, C. capsici, C. caricae, C. cariniferi, C. catinaense, C. caudasporum, C. caudatum, C. ochraceae, C. somersetense, C. zoysiae, C. chiangraiense, C. chlorophyti, C. citri-maximae, C. citricola, C. clavatum, C. cobbittiense, C. coccodes, C. coelogynes, C. coffeanum, C. colombiense, C. condaoense, C. crassipes, C. cymbidiicola, C. dacrycarpi, C. anthrisci, C. circinans, C. dematium, C. fructi, C. hemerocallidis, C. insertae, C. lineola, C. menispermi, C. quinquefoliae, C. sedi, C. spinaciae, C. shisoi, C. americae-borealis, C. antirrhinicola, C. bryoniicola, C. destructivum, C. fuscum, C. higginsianum, C. lentis, C. ocimi, C. panacicola, C. pisicola, C. tabaci, C. tanaceti, C. utrechtense, C. doitungense, C. duyunense, C. eryngiicola, C. euphorbiae, C. excelsum-altitudinum, C. feijoicola, C. fragariae, C. fructivorum, C. fusiforme, C. gigasporum, C. aenigma, C. aeschynomenes, C. alatae, C. alienum, C. aotearoa, C. asianum, C. camelliae, C. changpingense, C. chrysophilum, C. clidemiae, C. conoides, C. cordylinicola, C. endophyticum, C. fructicola, C. gloeosporioides, C. grevilleae, C. grossum, C. hebeiense, C. helleniense, C. henanense, C. horii, C. hystricis, C. jiangxiense, C. kahawae, C. makassarense, C. musae, C. nupharicola, C. perseae, C. proteae, C. psidii, C. queenslandicum, C. salsolae, C. siamense, C. syzygiicola, C. tainanense, C. theobromicola, C. ti, C. tropicale, C. viniferum, C. wuxiense, C. xanthorrhoeae, C. gossypii, C. cereale, C. echinochloae, C. eleusines, C. endophytum, C. eremochloae, C. falcatum, C. graminicola, C. hanaui, C. jacksonii, C. miscanthi, C. navitas, C. nicholsonii, C. paspali, C. guaranicola, C. hederiicola, C. hippeastri, C. hsienjenchang, C. hymenocallidicola, C. incarnatum, C. jasminigenum, C. jinshuiense, C. johnstonii, C. kakivorum, C. kinghornii, C. kniphofiae, C. lagenaria, C. lauri, C. ledebouriae, C. limonicola, C. lini, C. lobatum, C. magnisporum, C. brevisporum, C. cacao, C. liaoningense, C. magnum, C. merremiae, C. okinawense, C. panamense, C. metake, C. neosansevieriae, C. nicotianae, C. nigrum, C. novae-zelandiae, C. oncidii, C. bidentis, C. lindemuthianum, C. malvarum, C. orbiculare, C. sidae, C. tebeestii, C. trifolii, C. cattleyicola, C. cliviicola, C. dracaenophilum, C. musicola, C. orchidearum, C. piperis, C. plurivorum, C. sojae, C. vittalense, C. orchidophilum, C. pandanicola, C. parallelophorum, C. parsonsiae, C. phaseolorum, C. phormii, C. phyllachoroides, C. pisi, C. pseudoacutatum, C. pseudomajus, C. pyricola, C. pyrifoliae, C. radicis, C. rhexiae, C. rhombiforme, C. ricini, C. roseum, C. rusci, C. sambucicola, C. sansevieriae, C. serranegrense, C. sichuanense, C. sonchicola, C. bletillum, C. guizhouense, C. incanum, C. lilii, C. liriopes, C. riograndense, C. spaethianum, C. tofieldiae, C. verruculosum, C. spinosum, C. sublineola, C. sydowii, C. taiwanense, C. temperatum, C. torulosum, C. trichellum, C. tropicicola, C. aciculare, C. curcumae, C. truncatum, C. vietnamense, C. vignae, C. wanningense, C. watphraense, C. yulongense, C. yunnanense, most preferably C. lagenarium;
Botrytis species: B. aclada, B. allii, B. arisaemae, B. byssoidea, B. californica, B. caroliniana, B. cinerea, B. croci, B. cryptomeriae, B. elliptica, B. eucalypti, B. euroamericana, B. faba, B. fabae, B. fabiopsis, B. fragariae, B. fuckeliana, B. galanthina, B. hyacinthi, B. mali, B. paeoniae, B. pelargonii, B. porri, B. prunorum, B. pseudocinerea, B. sinoallii, B. sinoviticola, B. tulipae, most preferably B. cinerea;
Alternaria species: A. abutilonis, A. aconidiophora, A. allii, A. alstroemeriae, A. altcampina, A. alternariacida, A. anodae, A. argyranthemi, A. ascaloniae, A. atrans, A. azadirachtae, A. azukiae, A. beticola, A. bokurai, A. brassicae, A. brassicinae, A. broccoli-italicae, A. broussonetiae, A. bryophylli, A. capsicicola, A. caricicola, A. carthamicola, A. catalpae, A. catananches, A. celosiae, A. centaureae, A. cerasi, A. cerasidanica, A. cesenica, A. chenopodiicola, A. chlamydosporifera, A. chrysanthemi, A. citricancri, A. citrullicola, A. compacta, A. conidiophora, A. curvata, A. cyamopsidis, A. dactylidicola, A. daturicola, A. dennisii, A. deserticola, A. dianthi, A. doliconidium, A. echinaceae, A. eichhorniae, A. ellisii, A. ershadii, A. fasciculata, A. fimeti, A. forlicesenensis, A. fragaria, A. fulva, A. gansuensis, A. geophila, A. gossypina, A. grisea, A. grossulariae, A. hampshirensis, A. helianthiinficiens, A. heveae, A. humicola, A. hungarica, A. inflata, A. interrupta, A. ipomoeae, A. iranica, A. iridiaustralis, A. iridicola, A. iridis, A. italica, A. jacinthicola, A. jesenskae, A. kareliniae, A. kordkuyana, A. lawrencei, A. linariae, A. longissima, A. maritima, A. montanica, A. montsantina, A. murispora, A. napiformis, A. nelumbii, A. neoipomoeae, A. novae-guineensis, A. obtecta, A. ochroleuca, A. oxytropis, A. padwickii, A. paralinicola, A. parvicaespitosa, A. peglionii, A. peucedani, A. pharbitidis, A. physalidis, A. pipionipisi, A. pluriseptata, A. poaceicola, A. pobletensis, A. pomicola, A. populi, A. prasonis, A. pruni, A. pseudoventricosa, A. pulvinifungicola, A. quercicola, A. quercus, A. ranunculi, A. resedae, A. roseogrisea, A. rosicola, A. sanguisorbae, A. sennae, A. sesamicola, A. sidae, A. silybi, A. soliaridae, A. sorghicola, A. tamaricis, A. thalictrigena, A. thlaspis, A. thunbergiae, A. tillandsiae, A. tomato, A. tropaeoli, A. undulata, A. vaccinii, A. vanuatuensis, A. venezuelensis, A. viniferae, A. viticola, A. vitis, A. yaliinficiens, most preferably A. alternata;
Phaeosphaeria species: P. acaciae, P. ammophilae, P. ampeli, P. anchiala, P. arenaria, P. berlesei, P. breonadiae, P. calamicola, P. caricicola, P. caricinella, P. caricis, P. chiangraina, P. culmorum, P. cycadis, P. dennisiana, P. elongata, P. epicalamia, P. eustoma, P. fuckelii, P. fusispora, P. glyceriae-plicatae, P. graminis, P. halima, P. herpotrichoides, P. insignis, P. juncicola, P. juncina, P. juncophila, P. lindii, P. luctuosa, P. lunariae, P. lutea, P. lycopodina, P. marciensis, P. musae, P. nardi, P. nigrans, P. norfolcia, P. occulta, P. olivacea, P. orae-maris, P. oryzae, P. papayae, P. parvula, P. penniseti, P. phoenicicola, P. pleurospora, P. poagena, P. podocarpi, P. pontiformis, P. rousseliana, P. setosa, P. silenes-acaulis, P. silvatica, P. sinensis, P. sowerbyi, P. spartinae, P. spartinicola, P. thysanolaenicola, P. tofieldiae, P. triglochinicola, P. vagans, P. volkartiana, most preferably P. nodorum;
Macrophomina species: M euphorbiicola, M. pseudophaseolina, M. vaccinii, M. phaseolina, most preferably M. phaseolina;
Zymoseptoria species: Z. ardabiliae, Z. brevis, Z. crescenta, Z. halophila, Z. passerinii, Z. pseudotritici, Z. tritici, Z. verkleyi, most preferably Z. tritici;
Rhizoctonia species: R. alpina, R. bicornis, R. butinii, R. callae, R. carotae, R. endophytica, R. floccosa, R. fragariae, R. fraxini, R. fusispora, R. globularis, R. gossypii, R. muneratii, R. papayae, R. quercus, R. repens, R. rubi, R. silvestris, R. solani, most preferably R. solani;
Thanatephorus species: T. cucumeris, T. obscurus, T. ochraceus, T. pendulus, T. sasakii, most preferably T. cucumeris;
Uromyces species: U. acetosae, U. acuminatus, U. aemulus, U. aloes, U. alopecuri, U. alyxiae, U. anthyllidis, U. appendiculatus, U. ari-triphylli, U. betae, U. beticola, U. bidenticola, U. bulbinicola, U. caricis-sempervirentis, U. cestri, U. ciceris-arietini, U. clignyi, U. coloradensis, U. commelinae, U. coronatus, U. dactylidis, U. dianthi, U. dolicholi, U. durus, U. ehrhartae, U. eragrostidis, U. erythronii, U. euphorbiae, U. euphorbiae-corniculati, U. ficariae, U. gageae, U. galegae, U. gaubae, U. geranii, U. gladioli, U. goyazensis, U. halstedii, U. hawksworthii, U. hedysari-obscuri, U. hobsonii, U. holwayi, U. hordeinus, U. inaequialtus, U. intricatus, U. ixiae, U. japonicus, U. jonesii, U. junci, U. klotzschianus, U. laburni, U. lespedezae-procumbentis, U. limonii, U. lomandracearum, U. lupini, U. lycoctoni, U. magnusii, U. minor, U. musae, U. muscari, U. neotropicalis, U. novissimus, U. oaxacanus, U. orientalis, U. otaviensis, U. pedicellata, U. peglerae, U. phaseoli, U. pisi, U. pisi-sativi, U. plumbarius, U. polygoni-avicularis, U. probus, U. punctatus, U. reichertii, U. rumicis, U. salsolae, U. scaevolae, U. scillarum, U. scrophulariae, U. scutellatus, U. setariae-italicae, U. socius, U. sommerfeltii, U. sporobolicola, U. striatus, U. striolatus, U. strobilanthis, U. tenuicutis, U. transversalis, U. trifolii, U. trifolii-repentis, U. viciae-fabae, U. vignae, U. wedeliae, U. zygadeni, most preferably U. appendiculatus;
Puccinia species: P. aberrans, P. abnormis, P. abrupta, P. acetosae, P. achnatheri-sibirici, P. acroptili, P. actaeae-agropyri, P. actaeae-elymi, P. adenocauli, P. aegopodii, P. afra, P. agrophila, P. agropyrina, P. aizazii, P. albescens, P. albulensis, P. allii, P. alpina, P. amari, P. andropogonis, P. annularis, P. antirrhini, P. arachidis, P. arenariae, P. argentata, P. aridariae, P. arrhenatheri, P. arrhenathericola, P. artemisiae-keiskeanae, P. arthrocnemi, P. arundinariae, P. asarina, P. asparagi, P. asteris, P. atra, P. aucta, P. baccharidis, P. ballotiflora, P. balsamorrhizae, P. bardanae, P. bartholomaei, P. bassiae, P. berkheyicola, P. bistortae, P. boroniae, P. brachypodii, P. bromina, P. bupleuri, P. buxi, P. cacabata, P. calcitrapae, P. calthae, P. calthicola, P. calystegiae-soldanellae, P. canaliculata, P. cannacearum, P. cardui-pycnocephali, P. carduorum, P. caricina, P. caricis, P. caricis-montanae, P. caricis-stipatae, P. carissae, P. carthami, P. cenchri, P. cerinthes-agropyrina, P. cesatii, P. chardoniensis, P. chloridis, P. chondrillina, P. chrysanthemi, P. chrysosplenii, P. chunjii, P. circaeae, P. circumdata, P. clavata, P. cnici, P. cnici-oleracei, P. codyi, P. coleataeniae, P. colossea, P. commelinae, P. conoclinii, P. consimilis, P. convolvuli, P. coronata, P. coronati-agrostidis, P. coronati-brevispora, P. coronati-calamagrostidis, P. coronati-hordei, P. coronati-japonica, P. coronati-longispora, P. crandallii, P. crepidis-japonicae, P. crotonopsidis, P. crupinae, P. cumminsii, P. cyani, P. cygnorum, P. cymbopogonis, P. cynodontis, P. cyperi, P. dactylidina, P. dampierae, P. dianellae, P. dichondrae, P. dietelii, P. digitata, P. digraphidis, P. dimidipes, P. dioicae, P. dispersa, P. distincta, P. dolosa, P. drabae, P. dracunculina, P. durangensis, P. duthiae, P. elymi, P. emaculata, P. emiliae, P. esclavensis, P. eupatorii-columbiani, P. fergussonii, P. ferruginosa, P. firma, P. flavenscentis, P. fumosa, P. funkiae, P. galeniae, P. galiiuniversa, P. gansensis, P. gastrolobii, P. geitonoplesii, P. geranii-pilosi, P. gigantea, P. gilgiana, P. glechomatis, P. globosipes, P. gnaphaliicola, P. graminicola, P. graminis, P. grevilleae, P. haemodori, P. helianthi, P. hemerocallidis, P. heterogenea, P. heterospora, P. heucherae, P. hieracii, P. holcina, P. hordei, P. hordei-secalini, P. horiana, P. hydrocotyles, P. hypochoeridis, P. hysterium, P. impatientis, P. impedita, P. imposita, P. insolita, P. iridis, P. isiacae, P. jaceae, P. jasmini, P. jogashimensis, P. junci, P. justiciae, P. klugkistiana, P. knersvlaktensis, P. komarovii, P. kuehnii, P. kusanoi, P. lagenophorae, P. lantanae, P. lapsanae, P. lasiacidis, P. lateritia, P. levis, P. liberta, P. liliacearum, P. linkii, P. linosyridis-caricis, P. lippiivora, P. littoralis, P. lobata, P. lophatheri, P. loranthicola, P. ludwigii, P. luzulae, P. luzulae-maximae, P. lycii, P. lygodii, P. macra, P. maculosa, P. magnusiana, P. malvacearum, P. mariae-wilsoniae, P. marrubii, P. melampodii, P. melanocephala, P. mellifera, P. menthae, P. merrilliana, P. mesembryanthemi, P. mesnieriana, P. meyeri-albertii, P. mikaniae, P. millefolii, P. millegranae, P. miscanthi, P. miscanthidii, P. mixta, P. modiolae, P. monoica, P. montanensis, P. morata, P. morrisoni, P. morthieri, P. muehlenbeckiae, P. myrsiphylli, P. mysuruensis, P. nakanishikii, P. nepalensis, P. nigrescens, P. nishidana, P. nitida, P. novopanici, P. oahuensis, P. obscura, P. ocimi, P. oenanthes-stoloniferae, P. operta, P. otzeniani, P. oxalidis, P. oxyriae, P. paederiae, P. paludosa, P. pammelii, P. pampeana, P. pappophori, P. paradoxapoda, P. pascua, P. paspali, P. paspalina, P. patriniae, P. paullula, P. pazschkei, P. pelargonii-zonalis, P. penicillariae, P. pentstemonis, P. peperomiae, P. peradeniyae, P. perplexans, P. persistens, P. phlomidis, P. physalidis, P. pimpinellae, P. pittieriana, P. platyspora, P. poae-nemoralis, P. poarum, P. polygoni-amphibii, P. polysora, P. porri, P. pritzeliana, P. prostii, P. pseudodigitata, P. pseudomesnieriana, P. pseudostriiformis, P. psychotriae, P. punctata, P. punctiformis, P. purpurea, P. rapipes, P. recondita, P. rhagodiae, P. rhaphidophorae, P. rhei-undulati, P. ribesii-caricis, P. ribesii-diversicoloris, P. ribesii-pendulae, P. ribis, P. rufipes, P. rupestris, P. saccardoi, P. salihae, P. salviae, P. saxifragae, P. scirpi, P. scleriae, P. scorzonerae, P. senecionis, P. senecionis-acutiformis, P. septentrionalis, P. serpylli, P. sessilis, P. setariae, P. sherardiana, P. silphii, P. silvatica, P. similis, P. smilacis, P. sorghi, P. sparganioidis, P. spegazzinii, P. sporoboli, P. stipina, P. stobaeae, P. striiformis, P. striiformoides, P. stylidii, P. subalpina, P. substriata, P. suzutake, P. symphoricarpi, P. taeniatheri, P. tageticola, P. tanaceti, P. tatarinovii, P. tetragoniae, P. thaliae, P. thlaspeos, P. tillandsiae, P. tiritea, P. trebouxi, P. triticina, P. tubulosa, P. tulipae, P. tumidipes, P. turgida, P. uliginosa, P. unciniarum, P. unica, P. urbaniana, P. ursiniae, P. urticae-acutae, P. urticae-acutiformis, P. urticae-caricis, P. urticae-hirtae, P. urticae-inflatae, P. urticata, P. vaga, P. vaginatae, P. vernoniae-mollis, P. veronicae-longifoliae, P. versicolor, P. vexans, P. vincae, P. violae, P. virgata, P. virgaureae, P. wahlenbergiae, P. wiehei, P. windhoekensis, P. windsoriae, P. wolgensis, P. wyomensis, P. xanthii, P. xanthosiae, P. zoysiae, most preferably P. triticina;
Ustilago species: U. abaconensis, U. aeluropodis, U. affinis, U. agrostidis-palustris, U. airae-caespitosae, U. alcornii, U. alopecurivora, U. altilis, U. austro-africana, U. avenae, U. bouriqueti, U. brizae, U. bromina, U. bromivora, U. bullata, U. calamagrostidis, U. chloridis, U. coicis, U. comburens, U. corcontica, U. crameri, U. cruenta, U. curta, U. cynodontis, U. davisii, U. denotarisii, U. drakensbergiana, U. echinata, U. esculenta, U. filiformis, U. grandis, U. hordei, U. inaltilis, U. ixophori, U. jagei, U. kamerunensis, U. kummeri, U. levis, U. lituana, U. loliicola, U. longissima, U. maydis, U. milii, U. neocopinata, U. neyraudiae, U. nuda, U. nunavutica, U. pamirica, U. panici-gracilis, U. perennans, U. phrygica, U. pinguiculae, U. porosa, U. quitensis, U. salveii, U. scaura, U. schmidtiae, U. schroeteriana, U. scrobiculata, U. serpens, U. shanxiensis, U. shiraiana, U. siamensis, U. sieglingiae, U. sparsa, U. sparti, U. spermophora, U. sphaerogena, U. spinificis, U. sporoboli-indici, U. striiformis, U. syntherismae, U. tragana, U. tragopogonis-pratensis, U. trichophora, U. tritici, U. turcomanica, U. vetiveriae, U. vinosa, U. williamsii, U. xerochloae, most preferably U. segetum;
Pythium species: P. abappressorium, P. acanthicum, P. acrogynum, P. adhaerens, P. afertile, P. alternatum, P. amasculinum, P. anandrum, P. angustatum, P. aphanidermatum, P. apiculatum, P. apleroticum, P. aquatile, P. aristosporum, P. arrhenomanes, P. attrantheridium, P. baisense, P. barbulae, P. biforme, P. bifurcatum, P. brachiatum, P. brassicum, P. breve, P. buismaniae, P. burgundicum, P. butleri, P. campanulatum, P. camurandrum, P. canariense, P. capillosum, P. carolinianum, P. catenulatum, P. cederbergense, P. cedri, P. chondricola, P. coloratum, P. conidiophorum, P. contiguanum, P. cryptoirregulare, P. cucurbitacearum, P. cylindrosporum, P. cystogenes, P. debaryanum, P. deliense, P. destruens, P. diclinum, P. dimorphum, P. dissimile, P. dissotocum, P. echinogynum, P. echinulatum, P. emineosum, P. erinaceum, P. ershadii, P. flevoense, P. folliculosum, P. glomeratum, P. graminicola, P. grandisporangium, P. guangxiense, P. guiyangense, P. helicandrum, P. heteroogonium, P. heterothallicum, P. hydnosporum, P. hypogynum, P. indigoferae, P. inflatum, P. insidiosum, P. irregulare, P. iwayamai, P. jasmonium, P. junctum, P. kandovanense, P. kashmirense, P. kunmingense, P. longandrum, P. longipapillum, P. longisporangium, P. lucens, P. lutarium, P. lycopersicum, P. macrosporum, P. mamillatum, P. marinum, P. mastophorum, P. megalacanthum, P. middletonii, P. minus, P. monospermum, P. multisporum, P. myophilum, P. myriotylum, P. nagaii, P. nodosum, P. nunn, P. okanoganense, P. oligandrum, P. oopapillum, P. ornacarpum, P. ornamentatum, P. oryzicollum, P. pachycaule, P. paddicum, P. parvum, P. pectinolyticum, P. periilum, P. periplocum, P. perplexum, P. phragmiticola, P. phragmitis, P. pleroticum, P. plurisporium, P. polare, P. polymastum, P. porphyrae, P. prolatum, P. proliferatum, P. pulchrum, P. pyrilobum, P. pyrioosporum, P. quercum, P. radiosum, P. recalcitrans, P. regulare, P. rhizosaccharum, P. rishiriense, P. rostratifingens, P. rostratum, P. salinum, P. salpingophorum, P. schmitthenneri, P. scleroteichum, P. segnitium, P. selbyi, P. senticosum, P. solare, P. spiculum, P. sterilum, P. stipitatum, P. sukuiense, P. sulcatum, P. sylvaticum, P. takayamanum, P. tardicrescens, P. terrestris, P. torulosum, P. tracheiphilum, P. uncinulatum, P. urmianum, P. utonaiense, P. vanterpoolii, P. viniferum, P. violae, P. volutum, P. yorkensis, P. zingiberis, most preferably P. ultimum and P. irregulare;
Phytophthora species: P. abietivora, P. acerina, P. agathidicida, P. aleatoria, P. alni, P. alticola, P. amaranthi, P. amnicola, P. andina, P. aquimorbida, P. arecae, P. arenaria, P. asiatica, P. asparagi, P. attenuata, P. austrocedrae, P. balyanboodja, P. batemanensis, P. betacei, P. bilorbang, P. bisheria, P. bishii, P. boehmeriae, P. boodjera, P. borealis, P. botryosa, P. brassicae, P. cactorum, P. cacuminis, P. cajani, P. cambivora, P. capensis, P. capsici, P. captiosa, P. caryae, P. castaneae, P. castanetorum, P. chesapeakensis, P. chlamydospora, P. chrysanthemi, P. cichorii, P. cinnamomi, P. citricola, P. citrophthora, P. clandestina, P. cocois, P. colocasiae, P. condilina, P. constricta, P. cooljarloo, P. crassamura, P. cryptogea, P. cuyabensis, P. cyperi, P. dauci, P. drechsleri, P. elongata, P. erythroseptica, P. estuarina, P. europaea, P. fallax, P. flexuosa, P. fluvialis, P. foliorum, P. formosa, P. formosana, P. fragariae, P. fragariaefolia, P. frigida, P. gallica, P. gemini, P. gibbosa, P. glovera, P. gonapodyides, P. gondwanensis, P. gregata, P. hedraiandra, P. heveae, P. hibernalis, P. himalayensis, P. himalsilva, P. humicola, P. hydrogena, P. hydropathica, P. idaei, P. ilicis, P. inflata, P. insolita, P. intercalaris, P. intricata, P. inundata, P. ipomoeae, P. iranica, P. irrigata, P. katsurae, P. kernoviae, P. kwongonina, P. lactucae, P. lacustris, P. lateralis, P. lilii, P. litchii, P. litoralis, P. macilentosa, P. macrochlamydospora, P. meadii, P. medicaginis, P. megakarya, P. megasperma, P. melonis, P. mengei, P. mexicana, P. mirabilis, P. mississippiae, P. morindae, P. multivesiculata, P. multivora, P. nagaii, P. nemorosa, P. nicotianae, P. niederhauseri, P. niederhauserii, P. obscura, P. occultans, P. oleae, P. oreophila, P. ornamentata, P. pachypleura, P. palmivora, P. parasitica, P. parsiana, P. parvispora, P. phaseoli, P. pini, P. pinifolia, P. pisi, P. pistaciae, P. plurivora, P. pluvialis, P. polonica, P. porri, P. primulae, P. pseudocryptogea, P. pseudolactucae, P. pseudopolonica, P. pseudorosacearum, P. pseudosyringae, P. pseudotsugae, P. psychrophila, P. quercetorum, P. quercina, P. quininea, P. rhizophorae, P. richardiae, P. riparia, P. rosacearum, P. rubi, P. sansomeana, P. sinensis, P. siskiyouensis, P. sojae, P. stricta, P. sulawesiensis, P. syringae, P. tabaci, P. tentaculata, P. terminalis, P. thermophila, P. trifolii, P. tropicalis, P. tubulina, P. tyrrhenica, P. uliginosa, P. undulata, P. uniformis, P. urerae, P. vignae, P. virginiana, P. vulcanica, P. heterohybrida, P. incrassata, P. multiformis, P. pelgrandis, P. serendipita, most preferably P. infestans;
Plasmopara species: P. angelicae, P. angustiterminalis, P. australis, P. baudysii, P. chaerophylli, P. constantinescui, P. densa, P. destructor, P. epilobii, P. euphrasiae, P. geranii, P. geranii-sylvatici, P. halstedii, P. invertifolia, P. laserpitii, P. majewskii, P. megasperma, P. mei-foeniculi, P. muralis, P. nivea, P. obducens, P. pastinacae, P. penniseti, P. peucedani, P. pimpinellae, P. praetermissa, P. pusilla, P. siegesbeckiae, P. sii, P. skvortzovii, P. solidaginis, P. sphagneticolae, P. velutina, P. viticola, P. wildemaniana, P. wilsonii, most preferably P. viticola;
Pseudoperonospora species: P. cannabina, P. celtidis, P. cubensis, P. humuli, P. urticae, most preferably P. cubensis.

It is particularly advantageous that the plant health promotion compositions of the present invention are effective against pathogens of genus Fusarium as shown in the examples.

Correspondingly, the invention also provides a method for preventing, limiting or reducing a phytopathogenic fungal disease and/or for increasing the health of a plant, comprising applying an effective amount of the plant health promotion composition of the present invention to the plant, a part or propagation material thereof or to the soil where the plants are to grow. Thus, the plant health promotion composition can exercise its beneficial effects as described herein.

The invention is hereinafter described by examples and with reference to figures. Neither the figures nor the examples are intended to limit the scope of the invention.

### EXAMPLES

### Example 1: Nicotinic acid addition improves growth

Media

Preculture Medium: PX-125

The composition of PX-125 is listed in the table below. The components of the stock solution were dissolved in distilled water and either sterile filtered or autoclaved at 121° C., 1 bar overpressure for 60 min. The sterile solutions were stored either at room temperature or at 4° C. The antifoam agent was added to the main solution shortly before starting the autoclaving process. After mixing the stock solutions, the pH of the medium was set to 6.5 either with 25% (w/w) ammonia solution or 40% (w/w) phosphoric acid.

TABLE
Composition of the complex medium PX125 with the specification
for storage (room temperature (RT) or 4° C.) and sterilisation
method (sterile-filtered/autoclaved, s/a) of the stock solution.

| | | Concen- |
|---|---|---|
| | | tration |
| | | in the |
| | | medium |
| Stock solution | Component | [g/l] |
| Main solution (RT, a) | Citric acid monohydrate | 3.21 |
| | Dipotassium hydrogen | 1.00 |
| | phosphate | |
| | Ammonium sulfate | 1.07 |
| | Magnesium sulfate | 1.62 |
| | heptahydrate | |
| | Yeast extract | 5.00 |
| | Soy flour | 10.00 |
| | Antifoam | 0.2 |
| Calciumnitrate Tetrahydrate | Calcium nitrate tetrahydrate | 0.342 |
| (4° C., s) | | |
| Maltose (RT, a) | Maltose monohydrate | 363.00 |
| Vitamin solution | Thiamin hydrochloride | 0.005 |
| (4° C., s) | Nicotinic acid | 0.005 |
| | Riboflavin | 0.0002 |
| | Biotin | 0.00005 |
| | Calcium pantothenate | 0.001 |
| | Pyridoxin hydrochloride | 0.005 |
| | Vitamin B12 | 0.00005 |
| | Lipoic acid | 0.00005 |
| Trace element solution | Manganese sulfate | 0.013 |
| (4° C., s) | monohydrate | |
| | Copper sulfate pentahydrate | 0.0046 |
| | Sodium molybdate dihydrate | 0.0028 |
| | Iron sulfate monohydrate | 0.015 |
| | Citric acid monohydrate | 0.4 |

Main Culture Medium: Modified Poolman Medium

TABLE
Composition of the modified Poolman medium with the specification
for storage (room temperature (RT) or 4° C.) and sterilisation
method (sterile-filtered/autoclaved, s/a) of the stock solution.

| | | Concen- |
|---|---|---|
| | | tration |
| | | in the |
| | | medium |
| Stock solution | Component | [g/l] |
| Citric acid monohydrate with | Citric acid monohydrate | 3.6 |
| ammonium sulfate (RT, a) | Ammonium sulfate | 1.1 |
| Magnesium sulfate | Magnesium sulfate | 1.6 |
| heptahydrate (RT, a) | heptahydrate | |
| Calcium nitrate tetrahydrate | Calcium nitrate tetrahydrate | 0.342 |
| (4° C., s) | | |
| Maltose (RT, a) | Maltose syrup | 60.0 |
| MES-bufferbuffer pH = 6.5 | MES | 19.5 |
| (RT, s) | | |
| Antifoam solution (RT, a) | Antifoam | 0.2 |
| Dipotassium hydrogen- | Dipotassium hydrogen | 1.0 |
| phosphat (RT, s) | phosphate | |
| Trace element solution | Zink sulfate heptahydrate | 0.009 |
| (4° C., s) | Cobalt sulfate heptahydrate | 0.004 |
| | Copper sulfate pentahydrate | 0.004 |
| | Ammonium molybdate | 0.002 |
| | tetrahydrate | |
| Calcium-/manganese | Calcium chloride dihydrate | 0.066 |
| chloride solution (4° C., s) | Manganese chloride | 0.016 |
| Iron(II)-Iron(III)-chloride | Iron(II)-chloride | 0.005 |
| solution (−20° C., s) | Iron(III)-chloride | 0.005 |
| | hexahydrate | |
| Adenine (4° C., s) | Adenine | 0.01 |
| Guanine (4° C., s) | Guanine | 0.01 |
| Inosine (4° C., s) | Inosine | 0.005 |
| Xanthine (4° C., s) | Xanthine | 0.01 |
| Thymidine (4° C., s) | Thymidine | 0.005 |
| Uracile (4° C., s) | Uracile | 0.01 |
| Alanine (4° C., s) | Alanine | 0.24 |
| Arginine (4° C., s) | Arginine | 0.125 |
| Aspartic acid (4° C., s) | Aspartic acid | 0.42 |
| Cysteine (4° C., s) | Cysteine | 0.13 |
| Glutamic acid (4° C., s) | Glutamic acid | 0.5 |
| Glycine (4° C., s) | Glycine | 0.175 |
| Histidine (4° C., s) | Histidine | 0.15 |
| Isoleucine (4° C., s) | Isoleucine | 0.21 |
| Leucine (4° C., s) | Leucine | 0.475 |
| Lysine monohydrate | Lysine monohydrate | 0.49 |
| (4° C., s) | | |
| Methionine (4° C., s) | Methionine | 0.2 |
| Phenylalanine (4° C., s) | Phenylalanine | 0.275 |
| Proline (4° C., s) | Proline | 0.675 |
| Serine (4° C., s) | Serine | 0.34 |
| Threonine (4° C., s) | Threonine | 0.225 |
| Tryptophan (4° C., s) | Tryptophan | 0.05 |
| Tyrosine (4° C., s) | Tyrosine | 0.25 |
| Valine (4° C., s) | Valine | 0.325 |
| Ascorbic acid (4° C., s) | Ascorbic acid | 0.5 |
| Biotin (4° C., s) | Biotin | 0.00006 |
| Riboflavin (4° C., s) | Riboflavin | 0.001 |
| Orotic acid (4° C., s) | Orotic acid | 0.005 |
| Folic acid (4° C., s) | Folic acid | 0.001 |
| Inositol (4° C., s) | Inositol | 0.0002 |
| Vitamin solution | Nicotinic acid | 0.0011 |
| (4° C., s) | Calcium pantothenate | 0.001 |
| | p-Aminobenzoic acid | 0.01 |
| | Pyridoxamine | 0.006 |
| | dihydrochloride | |
| | Pyridoxine hydrochloride | 0.002 |
| | Vitamin B12 | 0.001 |
| | Thiamine hydrochloride | 0.001 |

The components of the stock solution were dissolved in distilled water and when required hydrochloric acid and potassium hydroxide were added to dissolve the vitamin, nucleotide and amino acid components.

The stock solution with the MES buffer was set to a pH of 6.5 with sodium hydroxide. The dipotassium hydrogen phosphate solution was set to a pH of 6.5 with phosphoric acid. The solutions of the Poolman medium were either autoclaved for 60 min at 121° C. and 1 bar overpressure or sterile filtered. The sterile solutions were stored either at room temperature or at 4° C. All the stock solutions were pipetted together, and the pH was set with 25% (w/w) ammonium solution and 40% (w/w) phosphoric acid to a pH of 6.5. In the end, the dipotassium hydrogen phosphate solution was added.

Cultivation Conditions

As pre-culture 30 ml of PX-125 medium was inoculated with 180 μl of a cryo culture vial. The cultivation took place for 24 h at 33° C. at the shaking frequency of 150 rpm and 25 mm shaking diameter in a 250 ml shake flask closed with silicon plugs.

For the main culture, the modified Poolman medium was then inoculated with 2% (v/v) of the pre-culture. The compositions of the modified Poolman medium were tested. As reference, the modified Poolman medium with the standard concentration of the vitamin solution was used. Then as second medium, the threefold concentration of the vitamin solution was used and third the standard vitamin solution with the threefold concentration of nicotinic acid was tested. The cultivation was conducted in a 48 round-well microtiterplate, with a filling volume of 800 μl, at 33° C. with a rotation speed of 1000 rpm, a shaking frequency of 3 mm. The plates were sealed with a sterile membrane allowing for gas transfer.

Offline samples were taken at the beginning and end of a cultivation at 66 h. The optical density of the culture medium was measured in a photometer at a wavelength of 600 nm. To remain in the linear range between 0.1 and 0.3 the samples were diluted with 0.9% (w/v) of sodium chloride solution, also used as blank. The OD values were corrected for evaporation by weighing the plates before and after the cultivation.

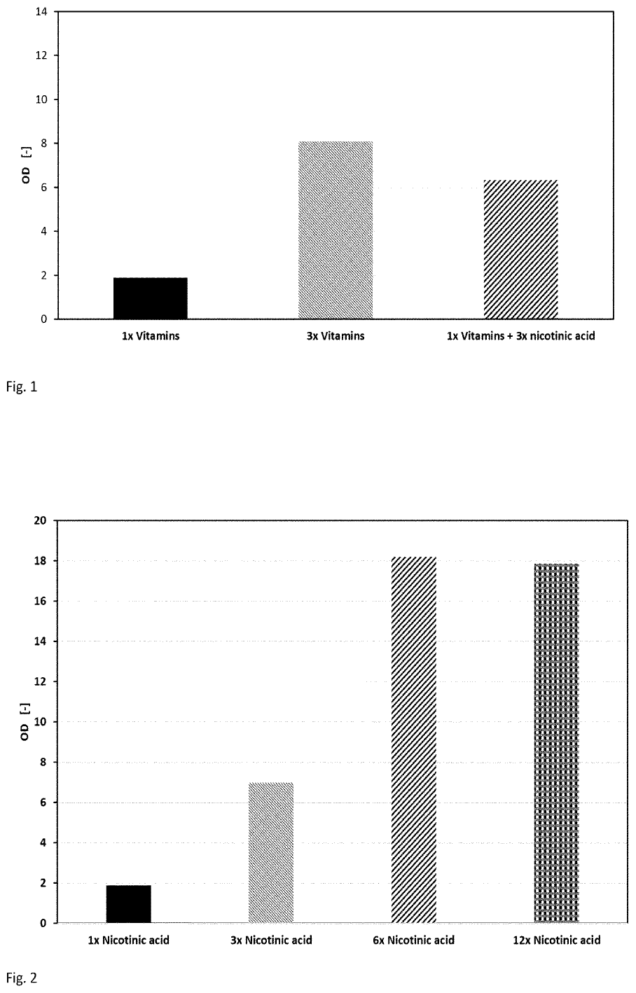

FIG. 1 shows the dependence of optical density (OD) on medium composition. Increase of nicotinic acid alone accounts for most of the increase in microorganism biomass, as evidenced by OD.

### Example 2: Nicotinic Acid Addition Improvement of Growth is Concentration Dependent

The cultivation was conducted according to method mentioned in example 1. The concentration of nicotinic acid was increased to 3×, 6× and 12× times of the initial concentration (see table example 1).

FIG. 2 shows the development of final OD depending on nicotinic acid concentration. With increasing nicotinic acid concentration the maximal bacterial growth is achieved.

### Example 3: Nicotinic Acid Addition Improves Fusaricidin Production

For the fusaricidin measurement of fermentation samples of example 2, 50 μl of culture broth was mixed together with 950 μl acetonitrile-water (1:1) mixture for extraction. The sample was treated for 30 min at 20° C. in an ultrasonic bath. The sample then was centrifuged for 5 min at 14000 rpm and the supernatant filtered into a HPLC vial for measurement. Fusaricidin concentration was determined by HPLC-UV-VIS as follows:
Column: Aqua C18, 250*4.6 mm (Phenomenex)
Pre-column: C18 Aqua
Temperature: 40
Flowrate: 1.00 mL/min
Injection volume: 2.0 μL
Detection: UV 200 nm
Maximal pressure: 400 bar
Eluent A: H₂O with 0.1% H3PO4
Eluent B: acetonitrile
Gradient:

| Time [min] | A [%] | B [%] | Flow [ml/min] |
|---|---|---|---|
| 0.0 | 770.0 | 330.0 | 1.00 |
| 9.0 | 660.0 | 440.0 | 1.00 |
| 12.0 | 0.00 | 100.0 | 1.00 |
| 18.0 | 70.00 | 3100.0 | 1.00 |

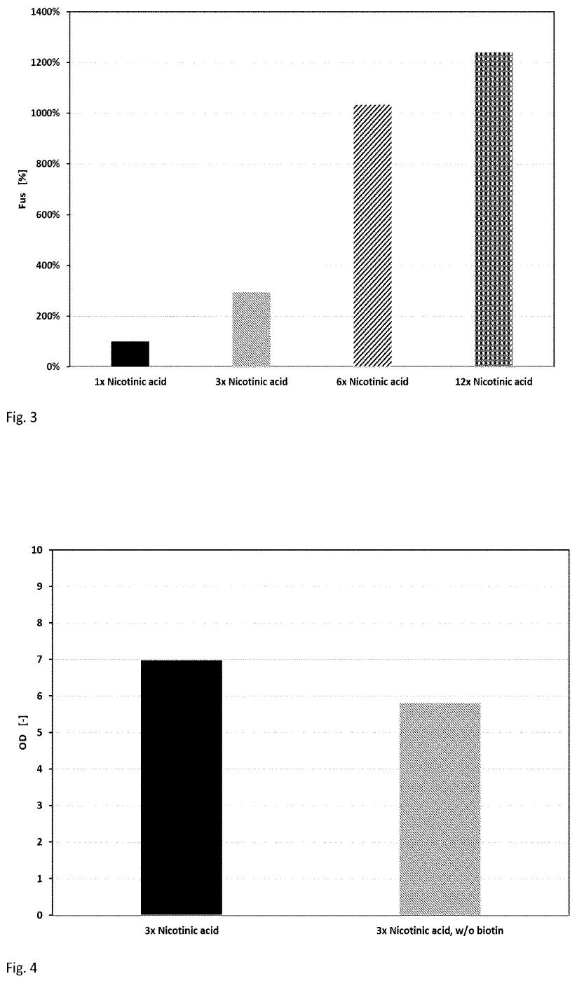

FIG. 3 shows the concentration of total fusaricidins A, B and D in the fermentation broth obtained in example 2. The fusaricidin concentration is increased at higher initial concentrations of nicotinic acid. At approximately 10 mg/l nicotinic acid the increase in fusaricidin concentration starts to level off.

### Example 4: Biotin Addition Improves Growth

The cultivation was conducted according to method mentioned in example 1. The concentration of nicotinic acid was increased to 3× times of the initial concentration and the experiment was conducted with medium with and without biotin (see table example 1).

FIG. 4 shows the final OD depending on biotin concentration. In the absence of biotin, the time for reaching maximum bacterial growth speed is markedly delayed and final biomass concentration is reduced.

### Example 5: Yeast Extract can be Reduced without Compromising Fusaricidin Content

Medium

Preculture Medium: PX-105

The composition of PX-105 is listed in the table. The components of the stock solution were dissolved in distilled water and either sterile filtered or autoclaved at 121° C., 1 bar overpressure for 60 min. The sterile solutions were stored either at room temperature or at 4° C. The antifoam agent was added to the main solution shortly before starting the autoclaving process. After mixing the stock solutions, the pH of the medium was set to 6.5 either with 25% (w/w) ammonia solution or 40% (w/w) phosphoric acid.

TABLE
Composition of the complex medium PX-105 with the specification
for storage (room temperature (RT) or 4° C.) and sterilisation
method (sterile-filtered/autoclaved, s/a) of the stock solution.

| | | Concen- |
|---|---|---|
| | | tration |
| | | in the |
| | | medium |
| Stock solution | Component | [g/l] |
| Main solution (RT, a) | Citric acid monohydrate | 3.21 |
| | Dipotassium hydrogen | 1.00 |
| | phosphate | |
| | Ammonium sulfate | 1.07 |
| | Magnesium sulfate | 1.62 |
| | heptahydrate | |
| | Yeast extract | 5.00 |
| | Soy flour | 10.00 |
| | Antifoam | 0.2 |
| Calcium nitrate tetrahydrate | Calcium nitrate tetrahydrate | 0.342 |
| (4° C., s) | | |
| Maltose (RT, a) | Maltose syrup (50%) | 120.00 |
| Vitamin solution | Thiamine hydrochloride | 0.005 |
| (4° C., s) | Nicotinic acid | 0.005 |
| | Riboflavin | 0.0002 |
| | Biotin | 0.00005 |
| | Calcium pantothenate | 0.001 |
| | Pyridoxine hydrochloride | 0.005 |
| | Vitamin B12 | 0.00005 |
| | Lipoic acid | 0.00005 |
| Trace element solution | Manganese sulfate | 0.013 |
| (4° C., s) | monohydrate | |
| | Copper sulfate pentahydrate | 0.0046 |
| | Sodium molybdate dihydrate | 0.0028 |
| | Iron sulfate monohydrate | 0.015 |
| | Citric acid monohydrate | 0.4 |

Main Culture Medium: PX-135

TABLE
Composition of the complex medium PX-135 with the specification
for storage (room temperature (RT) or 4° C.) and sterilisation
method (sterile-filtered/autoclaved, s/a) of the stock solution.
100 mM MES buffer was added to the medium.

| | | Concen- |
|---|---|---|
| | | tration |
| | | in the |
| | | medium |
| Stock solution | Component | [g/l] |
| Main solution (RT, a) | Citric acid monohydrate | 3.21 |
| | Dipotassium hydrogen | 1.00 |
| | phosphate | |
| | Ammonium sulfate | 1.07 |
| | Magnesium sulfate | 1.62 |
| | heptahydrate | |
| | Soy flour | 10.00 |
| | Antifoam | 0.2 |
| Calcium nitrate tetrahydrate | Calcium nitrate tetrahydrate | 0.342 |
| (4° C., s) | | |
| Maltose (RT, a) | Maltose syrup (50%) | 156.00 |
| Vitamin solution | Thiamine hydrochloride | 0.005 |
| (4° C., s) | Nicotinic acid | 0.005 |
| | Riboflavin | 0.0002 |
| | Biotin | 0.00005 |
| | Calcium pantothenate | 0.001 |
| | Pyridoxine hydrochloride | 0.005 |
| | Vitamin B12 | 0.00005 |
| | Lipoic acid | 0.00005 |
| Trace element solution | Manganese sulfate | 0.013 |
| (4° C., s) | monohydrate | |
| | Copper sulfate pentahydrate | 0.0046 |
| | Sodium molybdate dihydrate | 0.0028 |
| | Iron sulfate monohydrate | 0.015 |
| | Citric acid monohydrate | 0.4 |

Cultivation Conditions

As pre-culture, 30 ml of PX-105 medium was inoculated with 180 μl of a cryo culture vial. The cultivation took place for 24 h at 33° C. at the shaking frequency of 150 rpm and 25 mm shaking diameter in a 250 ml shake flask closed with breathable silicon plugs.

The main culture medium, PX-135, was then inoculated with 2% (v/v) of the pre-culture and used as reference. In a comparative medium 50 mg/l D/LL-methionine was added to the PX-135 medium. Further, the reference medium was complemented with 1.5 g/l, 3 g/l and 5 g/l yeast extract. In another set of flasks in addition to the aforementioned yeast concentrations, 200 mg/l DI-methionine was added. All these cultivations were conducted in 250 ml shake flask experiments with 30 ml filling volume and breathable silicon plugs for 48 h at 33° C. at the shaking frequency of 150 rpm and 25 mm shaking diameter.

Offline samples were taken at the start and end of a cultivation. The optical density of the culture medium was measured in a photometer at a wavelength of 600 nm. To remain in the linear range between 0.1 and 0.3 the samples were diluted with 0.9% (w/v) of sodium chloride solution, also used as blank.

For the fusaricidin measurement, 50 μl of culture broth was mixed together with 950 μl acetonitrile-water (1:1) mixture for extraction. The sample was treated for 30 min at 20° C. in an ultrasonic bath. The sample then was centrifuged for 5 min at 14000 rpm and the supernatant filtered into a HPLC vial for measurement. Fusaricidin concentration was determined as follows:

The analysis of fusaricidins was carried out by HPLC-UV-VIS as follows:
Column: Aqua C18, 250*4.6 mm (Phenomenex)
Pre-column: C18 Aqua
Temperature: 40
Flowrate: 1.00 mL/min
Injection volume: 2.0 μL
Detection: UV 200 nm
Maximal pressure: 400 bar
Eluent A: H₂O with 0.1% H3PO4
Eluent B: acetonitrile
Gradient:

| Time [min] | A [%] | B [%] | Flow [ml/min] |
|---|---|---|---|
| 0.0 | 80.0 | 20.0 | 1.00 |
| 20.0 | 50.0 | 50.0 | 1.00 |
| 30.0 | 0.00 | 100.0 | 1.00 |
| 40.0 | 0.00 | 100.0 | 1.00 |

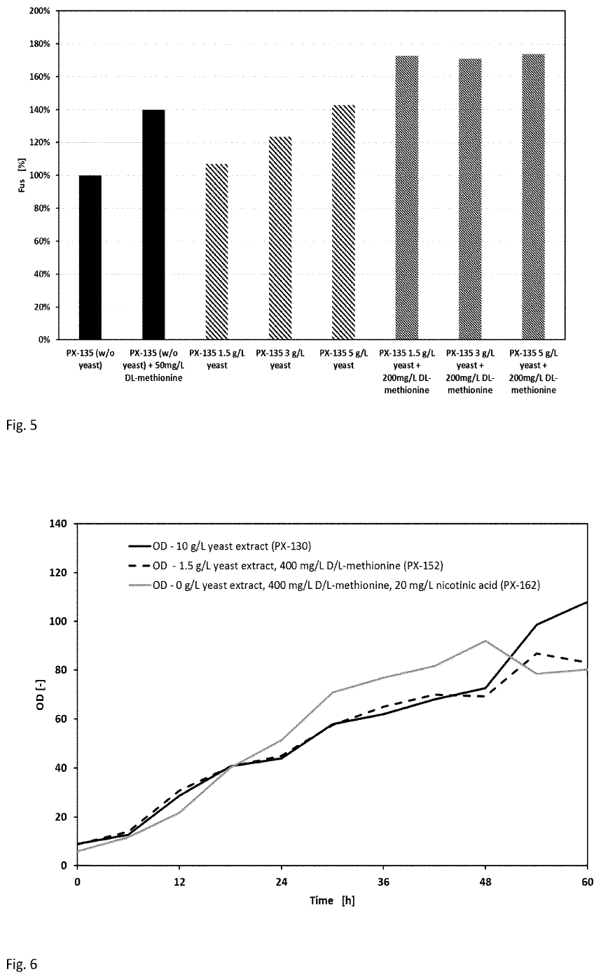

FIG. 5 shows the concentration of fusaricidins (total of fusaricidin A, B and D) in the fermentation broth after 48 h of fermentation. The fusaricidin concentration is highest in a medium with lowest initial yeast extract content and high initial concentration of DL-methionine.

### Example 6: Yeast Extract can be Reduced without Compromising Bacterial Growth

Medium

Preculture Medium: PX-125

The composition of PX-125 is listed in the table below. The components of the stock solution were dissolved in distilled water and either sterile filtered or autoclaved at 121° C., 1 bar overpressure for 60 m. The sterile solutions were stored either at room temperature or at 4° C. The antifoam agent was added to the main solution shortly before starting the autoclaving process. After mixing the stock solutions, the pH of the medium was set to 6.5 either with 25% (w/w) ammonia solution or 40% (w/w) phosphoric acid.

TABLE
Composition of the complex medium PX-125 with the specification
for storage (room temperature (RT) or 4° C.) and sterilisation
method (sterile-filtered/autoclaved, s/a) of the stock solution.

| | | Concen- |
|---|---|---|
| | | tration |
| | | in the |
| | | medium |
| Stock solution | Component | [g/l] |
| Main solution (RT, a) | Citric acid monohydrate | 3.21 |
| | Dipotassium hydrogen | 1.00 |
| | phosphate | |
| | Ammonium sulfate | 1.07 |
| | Magnesium sulfate | 1.62 |
| | heptahydrate | |
| | Yeast extract | 5.00 |
| | Soy flour | 10.00 |
| | Antifoam | 0.2 |
| Calcium nitrate tetrahydrate | Calcium nitrate tetrahydrate | 0.342 |
| (4° C., s) | | |
| Maltose (RT, a) | Maltose monohydrate | 63.00 |
| Vitamin solution | Thiamine hydrochloride | 0.005 |
| (4° C., s) | Nicotinic acid | 0.005 |
| | Riboflavin | 0.0002 |
| | Biotin | 0.00005 |
| | Calcium pantothenate | 0.001 |
| | Pyridoxine hydrochloride | 0.005 |
| | Vitamin B12 | 0.00005 |
| | Lipoic acid | 0.00005 |
| Trace element solution | Manganese sulfate | 0.013 |
| (4° C., s) | monohydrate | |
| | Zinc chloride | 0.004 |
| | Copper sulfate pentahydrate | 0.0046 |
| | Sodium molybdate dihydrate | 0.0028 |
| | Iron sulfate monohydrate | 0.015 |
| | Citric acid monohydrate | 0.4 |

Main Culture Medium: PX-130

TABLE
Composition of the complex medium PX-130 with the specification
for storage (room temperature (RT) or 4° C.) and sterilisation
method (sterile-filtered/autoclaved, s/a) of the stock solution.

| | | Concen- |
|---|---|---|
| | | tration |
| | | in the |
| | | medium |
| Stock solution | Component | [g/l] |
| Main solution (RT, a) | Citric acid monohydrate | 3.21 |
| | Dipotassium hydrogen | 1.00 |
| | phosphate | |
| | Ammonium sulfate | 1.07 |
| | Magnesium sulfate | 1.62 |
| | heptahydrate | |
| | Yeast extract | 10.00 |
| | Soy flour | 13.00 |
| | Antifoam | 0.2 |
| Calciumnitrate Tetrahydrate | Calcium nitrate tetrahydrate | 0.342 |
| (4° C., s) | | |
| Maltose (RT, a) | Maltose syrup (50%) | 156.00 |
| Vitamin solution | Thiamine hydrochloride | 0.005 |
| (4° C., s) | Nicotinic acid | 0.005 |
| | Riboflavin | 0.0002 |
| | Biotin | 0.00005 |
| | Calcium pantothenate | 0.001 |
| | Pyridoxine hydrochloride | 0.005 |
| | Vitamin B12 | 0.00005 |
| | Lipoic acid | 0.00005 |
| Trace element solution | Manganese sulfate | 0.013 |
| (4° C., s) | monohydrate | |
| | Copper sulfate pentahydrate | 0.0046 |
| | Sodium molybdate dihydrate | 0.0028 |
| | Iron sulfate monohydrate | 0.015 |
| | Citric acid monohydrate | 0.4 |

Main Culture Medium: PX-152

TABLE
Composition of the complex medium PX-152 with the specification
for storage (room temperature (RT) or 4° C.) and sterilisation
method (sterile-filtered/autoclaved, s/a) of the stock solution.

| | | Concen- |
|---|---|---|
| | | tration |
| | | in the |
| | | medium |
| Stock solution | Component | [g/l] |
| Main solution (RT, a) | Citric acid monohydrate | 3.21 |
| | Dipotassium hydrogen | 1.00 |
| | phosphate | |
| | Ammonium sulfate | 1.07 |
| | Magnesium sulfate | 1.62 |
| | heptahydrate | |
| | Yeast extract | 1.50 |
| | Soy flour | 13.00 |
| | Methionine | 0.4 |
| | Antifoam | 0.2 |
| Calcium nitrate tetrahydrate | Calcium nitrate tetrahydrate | 0.342 |
| (4° C., s) | | |
| Maltose (RT, a) | Maltose syrup (50%) | 156.00 |
| Vitamin solution | Thiamine hydrochloride | 0.005 |
| (4° C., s) | Nicotinic acid | 0.005 |
| | Riboflavin | 0.0002 |
| | Biotin | 0.00005 |
| | Calcium pantothenate | 0.001 |
| | Pyridoxine hydrochloride | 0.005 |
| | Vitamin B12 | 0.00005 |
| | Lipoic acid | 0.00005 |
| Trace element solution | Manganese sulfate | 0.013 |
| (4° C., s) | monohydrate | |
| | Copper sulfate pentahydrate | 0.0046 |
| | Sodium molybdate dihydrate | 0.0028 |
| | Iron sulfate monohydrate | 0.015 |
| | Citric acid monohydrate | 0.4 |

Main Culture Medium: PX-162

TABLE
Composition of the complex medium PX-162 with the specification
for storage (room temperature (RT) or 4° C.) and sterilisation
method (sterile-filtered/autoclaved, s/a) of the stock solution.

| | | Concen- |
|---|---|---|
| | | tration |
| | | in the |
| | | medium |
| Stock solution | Component | [g/l] |
| Main solution (RT, a) | Citric acid monohydrate | 3.21 |
| | Dipotassium hydrogen | 1.00 |
| | phosphate | |
| | Ammonium sulfate | 1.07 |
| | Magnesium sulfate | 1.62 |
| | heptahydrate | |
| | Soy flour | 13.00 |
| | Methionine | 0.4 |
| | Antifoam | 0.2 |
| Calcium nitrate tetrahydrate | Calcium nitrate tetrahydrate | 0.342 |
| (4° C., s) | | |
| Maltose (RT, a) | Maltose syrup (50%) | 156.00 |
| Vitamin solution | Thiamine hydrochloride | 0.005 |
| (4° C., s) | Nicotinic acid | 0.015 |
| | Riboflavin | 0.0002 |
| | Biotin | 0.00005 |
| | Calcium pantothenate | 0.001 |
| | Pyridoxine hydrochloride | 0.005 |
| | Vitamin B12 | 0.00005 |
| | Lipoic acid | 0.00005 |
| Trace element solution | Manganese sulfate | 0.013 |
| (4° C., s) | monohydrate | |
| | Copper sulfate pentahydrate | 0.0046 |
| | Sodium molybdate dihydrate | 0.0028 |
| | Iron sulfate monohydrate | 0.015 |
| | Citric acid monohydrate | 0.4 |

Cultivation Conditions

As pre-culture 110 ml of PX-125 medium was inoculated with 0.3% from a cryo culture vial. The cryo veils were heat-treated at 60° C. for 30 min. The cultivation took place for 24 h at 33° C. at the shaking frequency of 150 rpm and 25 mm shaking diameter in a 11 shake flask closed with breathable silicon plugs.

The main cultures were then inoculated with 2% (v/v) of the total volume. As reference PX-130 with 10 g/l yeast was used. In PX-152 the yeast concentration was reduced to 1.5 g/l with the addition of 400 mg/l methionine. Yeast extract could be omitted completely in the medium PX-162 through the addition of 400 mg/l methionine and increase of nicotinic acid to 0,015 g/l. All these cultivations were conducted in reactors with 12 L culture medium for 60 h at 33° C. The pH was set to 6.5 and adjusted with ammonium hydroxide or phosphoric acid. The dissolved oxygen was set to >20% by regulating stirrer speed (500-1200 rpm) and aeration (5-30 L/min).

Offline samples were taken at the beginning and end of a cultivation. The optical density of the culture medium was measured in a photometer at a wavelength of 600 nm. To remain in the linear range between 0.1 and 0.3 the samples were diluted with 0.9% (w/v) sodium chloride solution, also used as blank.

FIG. 6 shows the development of optical density depending on medium composition. Bacterial growth speed, as indicated by OD increase, is maintained in those media which comprise a reduced content of yeast extract and increased concentration of DL-methionine and nicotinic acid compared to a full yeast extract medium.

### Example 7: Yeast Extract can be Reduced without Compromising Fusaricidin Content

In the fermentations of example 6 fusaricidin concentrations were determined as described in example 5.

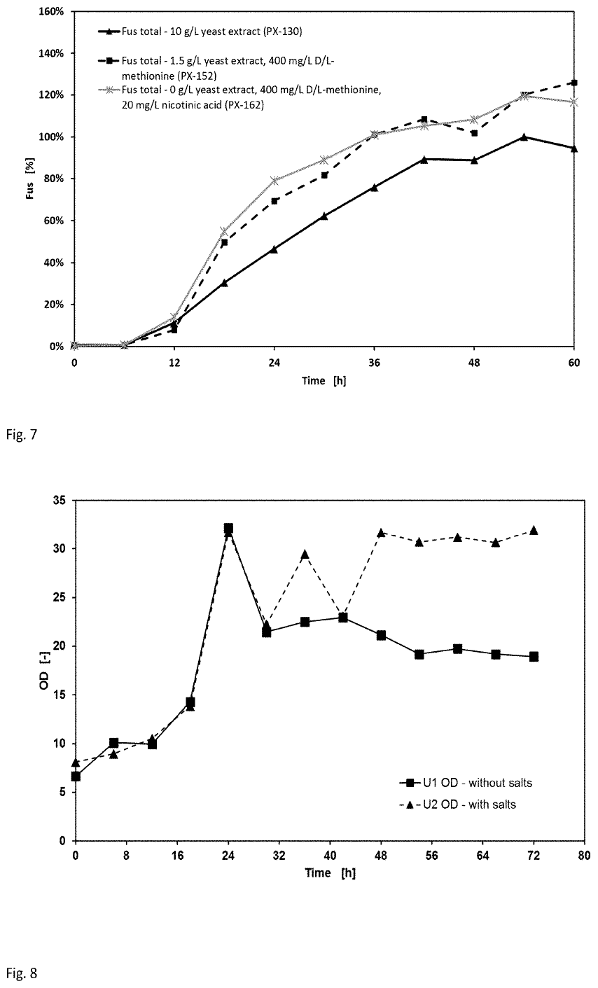

FIG. 7 shows the development of fusaricidin (total of fusaricidin A, B and D) concentration relative to maximum fusaricidin concentration for a yeast extract containing medium depending on medium composition. Fusaricidin concentration is higher in those media which comprise a reduced content of yeast extract and increased concentration of DL-methionine and nicotinic acid.

### Example 8: Salts Improve Bacterial Growth Rate During Fermentation

Medium

Preculture Medium: PX-176

The composition of PX-176 is listed in the table below. The components of the stock solution were dissolved in distilled water and either sterile filtered or autoclaved at 121° C., 1 bar overpressure for 60 min. The sterile solutions were stored either at room temperature or at 4° C. The antifoam agent was added to the main solution shortly before starting the autoclaving process. After mixing the stock solutions, the pH of the medium was set to 6.5 either with 25% (w/w) ammonia solution or 40% (w/w) phosphoric acid.

TABLE
Composition of the complex medium PX-176 with the specification
for storage (room temperature (RT) or 4° C.) and sterilisation
method (sterile-filtered/autoclaved, s/a) of the stock solution.

| | | Concen- |
|---|---|---|
| | | tration |
| | | in the |
| | | medium |
| Stock solution | Component | [g/l] |
| Main solution (RT, a) | Citric acid monohydrate | 3.21 |
| | Dipotassium hydrogen | 1.00 |
| | phosphate | |
| | Ammonium sulfate | 1.07 |
| | Magnesium sulfate | 1.62 |
| | heptahydrate | |
| | Yeast extract | 5.00 |
| | Soy flour | 10.00 |
| | Antifoam | 0.2 |
| Calcium nitrate tetrahydrate | Calcium nitrate tetrahydrate | 0.342 |
| (4° C., s) | | |
| Maltose (RT, a) | Maltose monohydrate | 30.00 |
| Vitamin solution | Thiamine hydrochloride | 0.005 |
| (4° C., s) | Nicotinic acid | 0.005 |
| | Riboflavin | 0.0002 |
| | Biotin | 0.00005 |
| | Calcium pantothenate | 0.001 |
| | Pyridoxine hydrochloride | 0.005 |
| | Vitamin B12 | 0.00005 |
| | Lipoic acid | 0.00005 |
| Trace element solution | Manganese sulfate | 0.013 |
| (4° C., s) | monohydrate | |
| | Copper sulfate pentahydrate | 0.0046 |
| | Sodium molybdate dihydrate | 0.0028 |
| | Iron sulfate monohydrate | 0.015 |
| | Citric acid monohydrate | 0.4 |

Main Culture Medium: PX-172

TABLE
Composition of the complex medium PX-172 with the specification
for storage (room temperature (RT) or 4° C.) and sterilisation
method (sterile-filtered/autoclaved, s/a) of the stock solution.

| | | Concen- |
|---|---|---|
| | | tration |
| | | in the |
| | | medium |
| Stock solution | Component | [g/l] |
| Main solution (RT, a) | Citric acid monohydrate | 3.21 |
| | Dipotassium hydrogen | 1.00 |
| | phosphate | |
| | Ammonium sulfate | 1.07 |
| | Magnesium sulfate | 1.62 |
| | heptahydrate | |
| | Soy flour | 13.00 |
| | D/L-methionine | 0.4 |
| | Antifoam | 0.2 |
| Calcium nitrate tetrahydrate | Calcium nitrate tetrahydrate | 0.342 |
| (4° C., s) | | |
| Maltose (RT, a) | Maltose syrup (50%) | 156.00 |
| | Nicotinic acid | 0.02 |
| | Biotin | 0.00015 |
| Trace element solution | Manganese sulfate | 0.013 |
| (4° C., s) | monohydrate | |
| | Copper sulfate pentahydrate | 0.0046 |
| | Sodium molybdate dihydrate | 0.0028 |
| | Iron sulfate monohydrate | 0.015 |
| | Citric acid monohydrate | 0.4 |

Cultivation Conditions

As pre-culture 80 ml of PX-176 medium was inoculated with 0.2% of a cryo culture vial. The cryo vials were heat-treated at 80° C. for 20 min. The cultivation took place for 24 h at 33° C. at the shaking frequency of 280 rpm and 25 mm shaking diameter in a 11 shake flask closed with breathable silicon plugs.

The main cultures were then inoculated with 2% (v/v) of the pre-culture. As reference PX-172 with trace element solution was used. In a parallel reactor the PX-172 medium was used without the addition of the trace element solution. All these cultivations were conducted in reactors with 1.1 L culture medium for 72 h at 33° C. The pH was set to 6.5 and adjusted with ammonium hydroxide or phosphoric acid. The dissolved oxygen (pO2) was set to >30% by regulating stirrer speed (400-1400 rpm) and aeration (18-180 L/h).

Offline samples were taken at the beginning and end of a cultivation. The optical density of the culture medium was measured in a photometer at a wavelength of 600 nm. To remain in the linear range between 0.1 and 0.3 the samples were diluted with 0.9% (w/v) sodium chloride solution, also used as blank.

FIG. 8 shows the development of OD depending on the presence of salts. Maximal bacterial growth, as indicted by optical density, is prolonged in the presence of salts.

### Example 9: Salts Improve Fusaricidin Production

In the fermentation according to example 8, fusaricidin concentrations were measured as described in example 3.

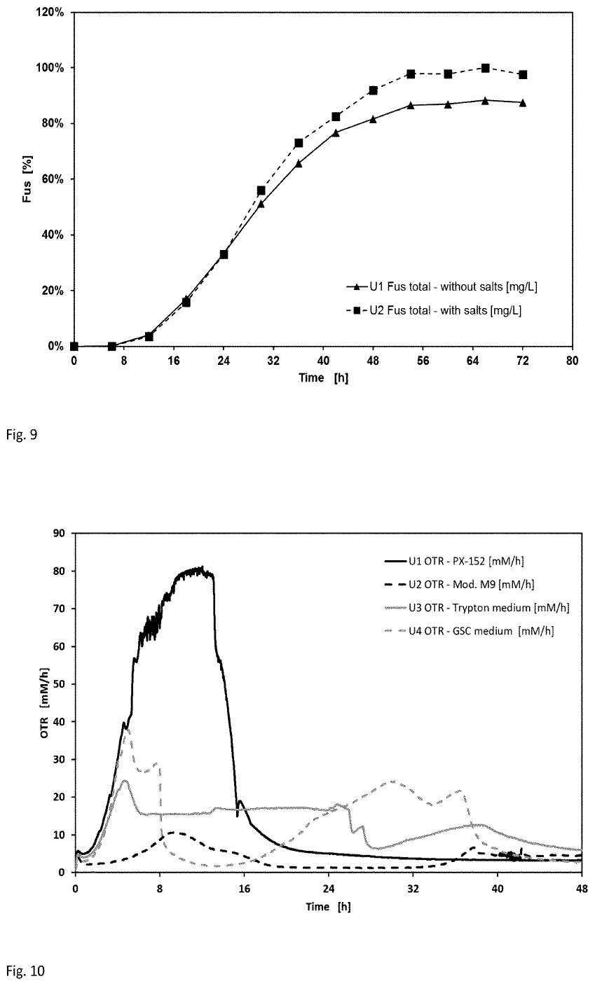

FIG. 9 shows the development of fusaricidin (total of fusaricidin A, B and D) concentration depending on the presence of salts. Fusaricidin concentration increases faster and reaches a higher maximum in the presence of salts, compared to a corresponding medium without addition of salts.

### Example 10: Comparison to Prior Art Media—Oxygen Demand During Fermentation

Medium

Preculture Medium: PX-48

The composition of PX-48 is listed in the table. The components of the stock solution were dissolved in distilled water and either sterile filtered or autoclaved at 121° C., 1 bar overpressure for 60 min. The sterile solutions were stored either at room temperature or at 4° C. The antifoam agent was added to the main solution shortly before starting the autoclaving process. After mixing the stock solutions, the pH of the medium was set to 6.5 either with 25% (w/w) ammonia solution or 40% (w/w) phosphoric acid.

TABLE
Composition of the complex medium PX-48 with the specification
for storage (room temperature (RT) or 4° C.) and sterilisation
method (sterile-filtered/autoclaved, s/a) of the stock solution.

| | | Concen- |
|---|---|---|
| | | tration |
| | | in the |
| | | medium |
| Stock solution | Component | [g/l] |
| Main solution (RT, a) | Citric acid monohydrate | 3.21 |
| | Monosodium phosphate | 2.72 |
| | Monopotassium phosphate | 5.95 |
| | Dipotassium hydrogen | 1.00 |
| | phosphate | |
| | Ammonium sulfate | 1.07 |
| | Diammonium phosphate | 2.95 |
| | Magnesium sulfate | 1.62 |
| | heptahydrate | |
| | Yeast extract | 5.00 |
| | Soy flour | 10.00 |
| | Antifoam | 0.2 |
| Calcium nitrate tetrahydrate | Calcium nitrate tetrahydrate | 0.342 |
| (4° C., s) | | |
| Maltose (RT, a) | Maltose monohydrate | 63.00 |
| Vitamin solution | Thiamine hydrochloride | 0.005 |
| (4° C., s) | Nicotinic acid | 0.005 |
| | Riboflavin | 0.0002 |
| | Biotin | 0.00005 |
| | Calcium pantothenate | 0.001 |
| | Pyridoxine hydrochloride | 0.005 |
| | Vitamin B12 | 0.00005 |
| | Lipoic acid | 0.00005 |
| Trace element solution | Manganese sulfate | 0.013 |
| (4° C., s) | monohydrate | |
| | Zinc chloride | 0.0040004 |
| | Ammonium chloride | 0.01 |
| | Copper sulfate pentahydrate | 0.0046 |
| | Sodium molybdate dihydrate | 0.0028 |
| | Calcium chloride | 0.002 |
| | Iron sulfate monohydrate | 0.015 |
| | Citric acid monohydrate | 0.4 |

Main Culture Medium: PX-152

TABLE
Composition of the complex medium PX-152 with the specification
for storage (room temperature (RT) or 4° C.) and sterilisation
method (sterile-filtered/autoclaved, s/a) of the stock solution.

| | | Concen- |
|---|---|---|
| | | tration |
| | | in the |
| | | medium |
| Stock solution | Component | [g/l] |
| Main solution (RT, a) | Citric acid monohydrate | 3.21 |
| | Dipotassium hydrogen | 1.00 |
| | phosphate | |
| | Ammonium sulfate | 1.07 |
| | Magnesium sulfate | 1.62 |
| | heptahydrate | |
| | Yeast extract | 1.55 |
| | Soy flour | 13.00 |
| | D/L-methionine | 0.4 |
| | Antifoam | 0.2 |
| Calcium nitrate tetrahydrate | Calcium nitrate tetrahydrate | 0.342 |
| (4° C., s) | | |
| Maltose (RT, a) | Maltose syrup (50%) | 156.00 |
| Vitamin solution | Thiamine hydrochloride | 0.005 |
| (4° C., s) | Nicotinic acid | 0.005 |
| | Riboflavin | 0.0002 |
| | Biotin | 0.00005 |
| | Calcium pantothenate | 0.001 |
| | Pyridoxine hydrochloride | 0.005 |
| | Vitamin B12 | 0.00005 |
| | Lipoic acid | 0.00005 |
| Trace element solution | | |
| (4° C., s) | Manganese sulfate | 0.013 |
| | monohydrate | |
| | Copper sulfate pentahydrate | 0.0046 |
| | Sodium molybdate dihydrate | 0.0028 |
| | Iron sulfate monohydrate | 0.015 |
| | Citric acid monohydrate | 0.4 |

TABLE
Composition of the modified M9 medium. All components,
except glucose, were mixed together and autoclaved
and stored at room temperature. The pH was set to 6.5
with either phosphoric acid or ammonia solution.

| Component | Concentration in the medium [g/l] |
|---|---|
| Na2HPO4 | 6 |
| KH2PO4 | 3 |
| NaCl | 0.5 |
| NH4Cl | 1 |
| MgSO4 × 7 H2O | 0.25 |
| CaCl2 | 0.011 |
| Glucose | 40 |
| FeSO4 | 0.152 |
| MnCl2 | 0.00013 |

TABLE
Composition of the complex medium Tryptone broth medium. All
components, except for iron sulfate, were mixed together
autoclaved and stored at room temperature. The pH was set
to 6.5 with either phosphoric acid or ammonia solution.

| Component | Concentration in the medium [g/l] |
|---|---|
| Tryptone | 10 |
| NaCl | 5 |
| Saccharose | 40 |
| MgSO4 × 7 H2O | 0.325 |
| ZnSO4 | 0.0288 |
| FeCl3 × 6 H2O | 0.0136 |

TABLE
Composition of the GSC medium. All components, except for
the iron sulfate and glucose solutions, were mixed together,
autoclaved and stored at room temperature. The pH was set
to 6.5 with either phosphoric acid or ammonia solution.

| Component | Concentration in the medium [g/l] |
|---|---|
| Glucose | 20 |
| Starch | 20 |
| (NH4)2SO4 | 20 |
| Yeast extract | 10 |
| K2HPO4 | 2.6 |
| FeSO4•7H2O | 0.1 |
| MgSO4•7H2O | 0.5 |
| NaCl | 0.25 |
| CaCO3 | 9 |

Cultivation Conditions

As pre-culture, 85 ml of PX-48 medium was inoculated with a 48 h grown Paenibacillus polymyxa M1 which derived from a ISP2 cultivation plate:

TABLE
Composition of the ISP2 agar medium. All components were mixed
together, autoclaved and stored at room temperature.

| Component | Concentration in the medium [g/l] |
|---|---|
| Yeast extract | 4 |
| Dextrose | 4 |
| Malt extract | 10 |
| Agar | 15 |
| Water | Add 1 L |

The liquid cultivation in PX-48 took place for 24 h at 33° C. at the shaking frequency of 280 rpm and 25 mm shaking diameter in a 11 shake flask closed with breathable silicon plugs. The main cultures were then inoculated with 2% (v/v) of the pre-culture. The performance of PX-152 medium was evaluated with other common cultivation medium mentioned in literature (Modified M9 medium, Ryu et al. 2019; tryptone medium, Raza et al. 2010; GSC medium, Nyu et al. 2013) for Paenibacillus cultivation. All these cultivations were conducted in reactors with 1 L culture medium for 72 h at 33° C. The pH was set to 6.5 and adjusted with ammonium hydroxide or phosphoric acid. The dissolved oxygen was set to >30% by regulating stirrer speed (400-1400 rpm) and aeration (18-180 sl/h).

Offline samples were taken at the beginning and end of a cultivation. The optical density of the culture medium was measured in a photometer at a wavelength of 600 nm. To remain in the linear range between 0.1 and 0.3 the samples were diluted with 0.9% (w/v) of sodium chloride solution, also used as blank.

For the fusaricidin measurement, 50 μl of culture broth was mixed together with 950 μl acetonitrile-water (1:1) mixture for extraction. The sample was treated for 30 min at 20° C. in an ultrasonic bath. The sample then was centrifuged for 5 min at 14000 rpm and the supernatant filtered into a HPLC vial for measurement. Fusaricidin concentration was measured as described in example 55.

FIG. 10 shows the development of oxygen transfer rate (OTR) depending on medium composition. Maximal bacterial metabolic activity, as indicted by OTR, is highest for a medium of the present invention compared to prior art media when normalising for sugar source concentration.

### Example 11: Comparison to Prior Art Media—Fusaricidin Yield

In the fermentation according to example 10, fusaricidin concentrations were measured as described in example 5.

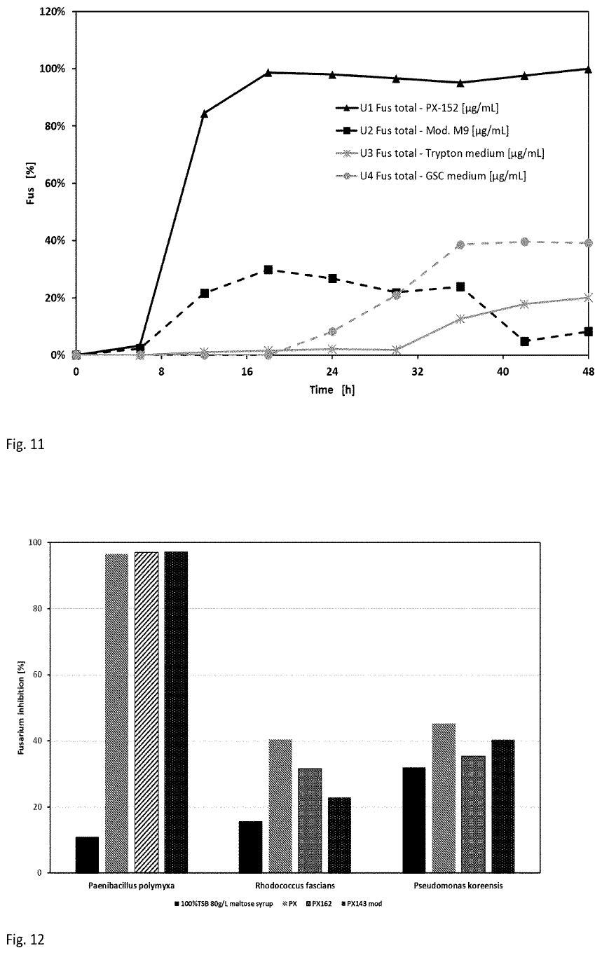

FIG. 11 shows the development of fusaricidin concentration (total of fusaricidins A, B and D) depending on medium composition. Fusaricidin concentration is highest for a medium of the present invention compared to prior art media when normalising for sugar source concentration.

### Example 12: Efficacy of Plant Health Promotion Compositions Against Fusarium

TABLE
Composition of the complex medium PX with the specification for
storage (room temperature (RT) or 4° C.) and sterilisation
method (sterile-filtered/autoclaved, s/a) of the stock solution.

| | | Concen- |
|---|---|---|
| | | tration |
| | | in the |
| | | medium |
| Stock solution | Component | [g/l] |
| Main solution (RT, a) | Citric acid monohydrate | 3.21 |
| | Dipotassium hydrogen | 1.00 |
| | phosphate | |
| | Ammonium sulfate | 1.07 |
| | Magnesium sulfate | 1.62 |
| | heptahydrate | |
| | Yeast extract | 5.00 |
| | Antifoam | 0.2 |
| Calcium nitrate tetrahydrate | Calcium nitrate tetrahydrate | 0.342 |
| (4° C., s) | | |
| Maltose (RT, a) | Maltose monohydrate | 63.00 |
| Vitamin solution | Thiamine hydrochloride | 0.005 |
| (4° C., s) | Nicotinic acid | 0.005 |
| | Riboflavin | 0.0002 |
| | Biotin | 0.00005 |
| | Calcium pantothenate | 0.001 |
| | Pyridoxine hydrochloride | 0.005 |
| | Vitamin B12 | 0.00005 |
| | Lipoic acid | 0.00005 |
| Trace element solution | | |
| (4 ° C., s) | Manganese sulfate | 0.013 |
| | monohydrate | |
| | Copper sulfate pentahydrate | 0.0046 |
| | Sodium molybdate dihydrate | 0.0028 |
| | Iron sulfate monohydrate | 0.015 |
| | Citric acid monohydrate | 0.4 |

TABLE
Composition of the complex medium PX-143 mod with the specification
for storage (room temperature (RT) or 4° C.) and sterilisation
method (sterile-filtered/autoclaved, s/a) of the stock solution.
100 mM MES buffer was added to the medium.

| | | Concen- |
|---|---|---|
| | | tration |
| | | in the |
| | | medium |
| Stock solution | Component | [g/l] |
| Main solution (RT, a) | Citric acid monohydrate | 3.21 |
| | Dipotassium hydrogen | 1.00 |
| | phosphate | |
| | Ammonium sulfate | 1.07 |
| | Magnesium sulfate | 1.62 |
| | heptahydrate | |
| | Yeast extract | 10.00 |
| | Soy flour | 13.00 |
| | Antifoam | 0.2 |
| Calcium nitrate tetrahydrate | Calcium nitrate tetrahydrate | 0.342 |
| (4° C., s) | | |
| Maltose (RT, a) | Maltose syrup (50%) | 40.00 |
| Vitamin solution | Thiamine hydrochloride | 0.005 |
| (4° C., s) | Nicotinic acid | 0.005 |
| | Riboflavin | 0.0002 |
| | Biotin | 0.00005 |
| | Calcium pantothenate | 0.001 |
| | Pyridoxine hydrochloride | 0.005 |
| | Vitamin B12 | 0.00005 |
| | Lipoic acid | 0.00005 |
| Trace element solution | | |
| (4° C., s) | Manganese sulfate | 0.013 |
| | monohydrate | |
| | Copper sulfate pentahydrate | 0.0046 |
| | Sodium molybdate dihydrate | 0.0028 |
| | Iron sulfate monohydrate | 0.015 |
| | Citric acid monohydrate | 0.4 |

TABLE
Composition of the complex medium PX-162 with the specification
for storage (room temperature (RT) or 4° C.) and sterilisation
method (sterile-filtered/autoclaved, s/a) of the stock solution.
100 mM MES buffer was added to the medium.

| | | Concen- |
|---|---|---|
| | | tration |
| | | in the |
| | | medium |
| Stock solution | Component | [g/l] |
| Main solution (RT, a) | Citric acid monohydrate | 3.21 |
| | Dipotassium hydrogen | 1.00 |
| | phosphate | |
| | Ammonium sulfate | 1.07 |
| | Magnesium sulfate | 1.62 |
| | heptahydrate | |
| | Soy flour | 10.00 |
| | Methionine | 0.4 |
| | Antifoam | 0.2 |
| Calcium nitrate tetrahydrate | Calcium nitrate tetrahydrate | 0.342 |
| (4° C., s) | | |
| Maltose (RT, a) | Maltose syrup (50%) | 156.00 |
| Vitamin solution | Thiamine hydrochloride | 0.005 |
| (4° C., s) | Nicotinic acid | 0.015 |
| | Riboflavin | 0.0002 |
| | Biotin | 0.00005 |
| | Calcium pantothenate | 0.001 |
| | Pyridoxine hydrochloride | 0.005 |
| | Vitamin B12 | 0.00005 |
| | Lipoic acid | 0.00005 |
| Trace element solution | | |
| (4° C., s) | Manganese sulfate | 0.013 |
| | monohydrate | |
| | Copper sulfate pentahydrate | 0.0046 |
| | Sodium molybdate dihydrate | 0.0028 |
| | Iron sulfate monohydrate | 0.015 |
| | Citric acid monohydrate | 0.4 |

TABLE
Composition of the complex medium
TSB medium set to a pH of 6.5.

| Component | Concentration in the medium [g/l] |
|---|---|
| Pancreatic digest of casein | 15 |
| Pancreatic digest of soybean (soyton) | 5 |
| Sodium chloride | 5 |
| Maltose syrup (50%) | 40 |

TABLE
Composition of the complex medium PBS medium.

| Component | Concentration in the medium [M] |
|---|---|
| Phosphate buffer | 0.01 |
| Potassium chloride | 0.0027 |
| Sodium chloride | 0.137 |

TABLE
Composition of the complex medium MPG medium.

| Component | Concentration in the medium [g/l] |
|---|---|
| Glucose | 14.6 |
| Mycopeptone | 7.1 |
| Yeast extract | 1.4 |

Cultivation of Bacteria and Sample Preparation for Fungal Assay

A 48 deep well plate with 6 ml volume was filled with 0.5 ml with microbial growth medium (tryptic soy broth, PX, PX-143 mod, PX-162). The various media were inoculated using cryopreserved bacteria (0.6% v/v). Each condition was replicated four times. An initial OD measurement at 600 nm was conducted using the corresponding non-inoculated medium as blank. Bacteria were cultivated for three days at 28° C. with shaking at 190 rpm and 80% humidity. At the end of the cultivation, bacterial growth was evaluated by measuring OD₆₀₀. Cultivation broths were centrifuged for 10 min at 4500 rpm to remove biomass. Further broth clarification was achieved by filtering 200 μL of the supernatant through a 96 well 0.2 μm filter membrane plate. The filtrate used for the fungal assay was obtained by centrifugation of the plates at 4500 rpm for 10 min.

Preparation of Fungal Spores

The fungal spores used in the screening assay were from Fusarium graminearum and Botrytis cinerea. For spore harvest, 5 ml PBS buffer were added to each fungal plate and the biomass was gently scraped off using a sterilized spreader rod. To remove the mycelium, the spore suspensions were filtered through a 0.4 μm filter membrane. For cryopreservation, spores were resuspended in PBS buffer with 10% glycerol and 7% L-proline. The spore concentration was adjusted to 5.3×10⁶ spores/ml. Cryo vials were stored at −20° C. for 24 h and afterwards at −80° C.

Set Up of Fungal Assay

The cryopreserved fungal inoculum was thawed for 30 min at room temperature and added to a sterile flask containing 140 ml fungal growth medium (MPG). The previously prepared supernatants of the bacterial cultivations were filled into a 96-well microtiter plate with each well containing 15 μl. As controls, 15 μl of each bacterial cultivation medium were pipetted into the plate. Then, 135 μl of the fungal inoculum were added to each well. The plates were incubated at room temperature in the dark for three (F. graminearum) or seven days (B. cinerea). To determine fungal inhibition, OD₆₂₀ was measured at incubation end and calculated as follows:

$$Fungal inhibition [ \% ] = \frac{( {OD}_{620} ( medium control ) - {OD}_{620} ( sample ) )}{{OD}_{620} ( medium control )} \times 100$$

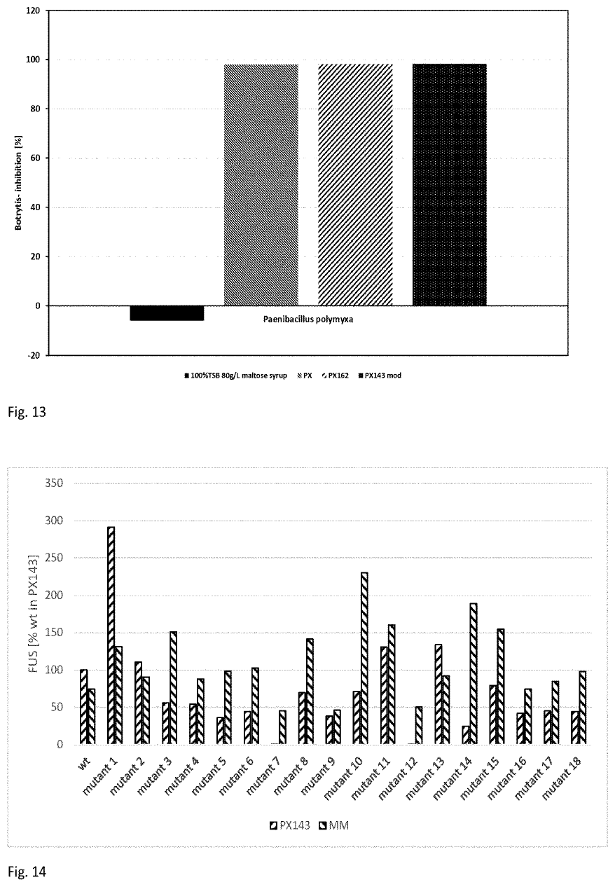

FIG. 12 shows the efficacy of cell-free culture filtrate against Fusarium graminearum after cultivation of various plant health promoting microorganisms. Generally, efficacy is improved for filtrates obtained after cultivation of those organisms in media according to the present invention.

FIG. 13 shows the efficacy of cell-free culture filtrate against Botrytis cinerea after cultivation of Paenibacillus. Generally, efficacy is improved for filtrates obtained after cultivation in media according to the present invention.

### Example 13: Broad Applicability of the Minimal Medium of the Present Invention

Medium

Preculture Medium: PX-79

The composition of PX-79 is listed in the table. The components of the stock solution were dissolved in distilled water and either sterile filtered or autoclaved at 121° C., 1 bar overpressure for 60 mi. The sterile solutions were stored either at room temperature or at 4° C. The antifoam agent was added to the main solution shortly before starting the autoclaving process. After mixing the stock solutions, the pH of the medium was set to 6.5 either with 25% (w/w) ammonia solution or 40% (w/w) phosphoric acid.

TABLE
Composition of the complex medium PX-79 with the specification for
storage (room temperature (RT) or 4° C.) and sterilisation
method (sterile-filtered/autoclaved, “s”/“a”) of the stock solution.

| | | Concen- |
|---|---|---|
| | | tration |
| | | in the |
| | | medium |
| Stock solution | Component | [g/l] |
| Main solution (RT, a) | Citric acid monohydrate | 3.21 |
| | Dipotassiumhydrogen | 1.00 |
| | phosphate | |
| | Ammonium sulfate | 1.07 |
| | Magnesium sulfate | 1.62 |
| | Heptahydrate | |
| | Yeast extract | 5.00 |
| | Soy flour | 10.00 |
| | Methionine | 0.4 |
| | Antifoam | 0.2 |
| Calciumnitrate Tetrahydrate | Calcium nitrate tetrahydrat | 0.342 |
| (4° C., s) | | |
| Maltose (RT, a) | Maltose syrup (50%) | 120.00 |
| Vitamin solution | Thiamin hydrochloride | 0.005 |
| (4° C., s) | Nicotinic acid | 0.005 |
| | Riboflavin | 0.0002 |
| | Biotin | 0.00005 |
| | Calcium pantothenate | 0.001 |
| | Pyridoxin hydrochlorid | 0.005 |
| | Vitamin B12 | 0.00005 |
| | Lipoic acid | 0.00005 |
| Trace element solution | | |
| (4° C., s) | Manganese sulfate | 0.013 |
| | monohydrate | |
| | Copper sulfate pentahydrate | 0.0046 |
| | Sodium molybdate dihydrate | 0.0028 |
| | Iron sulfate monohydrate | 0.015 |
| | Citric acid monohydrate | 0.4 |

Main Culture Medium: PX-143

TABLE
Composition of the complex medium PX-143 with the specification for
storage (room temperature (RT) or 4° C.) and sterilisation
method (sterile-filtered/autoclaved, “s”/“a”) of the stock solution.

| | | Concen- |
|---|---|---|
| | | tration |
| | | in the |
| | | medium |
| Stock solution | Component | [g/l] |
| Main solution (RT, a) | Citric acid monohydrate | 3.21 |
| | Dipotassiumhydrogen | 1.00 |
| | phosphate | |
| | Ammonium sulfate | 1.07 |
| | Magnesium sulfate | 1.62 |
| | Heptahydrate | |
| | MES-Puffer | 19.52 |
| | Yeast extract | 3.00 |
| | Soy flour | 13.00 |
| | Methionine | 0.4 |
| | Antifoam | 0.2 |
| Calciumnitrate Tetrahydrate | Calcium nitrate tetrahydrat | 0.342 |
| (4° C., s) | | |
| Maltose (RT, a) | Maltose syrup (50%) | 156.00 |
| Vitamin solution | Thiamin hydrochloride | 0.005 |
| (4° C., s) | Nicotinic acid | 0.015 |
| | Riboflavin | 0.0002 |
| | Biotin | 0.00005 |
| | Calcium pantothenate | 0.001 |
| | Pyridoxin hydrochlorid | 0.005 |
| | Vitamin B12 | 0.00005 |
| | lipoic acid | 0.00005 |
| Trace element solution | | |
| (4° C., s) | Manganese sulfate | 0.013 |
| | monohydrate | |
| | Copper sulfate pentahydrate | 0.0046 |
| | Sodium molybdate dihydrate | 0.0028 |
| | Iron sulfate monohydrate | 0.015 |
| | Citric acid monohydrate | 0.4 |

Main Culture Medium: Minimal medium

TABLE
Composition of the minimal medium. All components,
except glucose, were mixed together and autoclaved
and stored at room temperature. The pH was set to
6.5 with either phosphoric acid or ammonia solution.

| | | Concen- |
|---|---|---|
| | | tration |
| | | in the |
| | | medium |
| Stock solution | Component | [g/l] |
| Main solution (RT, a) | Citric acid monohydrate | 3.21 |
| | Dipotassiumhydrogen | 1.00 |
| | phosphate | |
| | Ammonium sulfate | 5 |
| | Magnesium sulfate | 1.62 |
| | Heptahydrate | |
| | MES-Puffer | 39 |
| | Antifoam | 0.2 |
| Calciumnitrate Tetrahydrate | Calcium nitrate tetrahydrat | 0.342 |
| (4° C., s) | | |
| Maltose (RT, a) | Maltose syrup (50%) | 156.00 |
| Vitamin solution | Thiamin hydrochloride | 0.005 |
| (4° C., s) | Nicotinic acid | 0.005 |
| | Riboflavin | 0.0002 |
| | Biotin | 0.00005 |
| | Calcium pantothenate | 0.001 |
| | Pyridoxin hydrochlorid | 0.005 |
| | Vitamin B12 | 0.00005 |
| | Lipoic acid | 0.00005 |
| Trace element solution | | |
| (4° C., s) | Manganese sulfate | 0.013 |
| | monohydrate | |
| | Copper sulfate pentahydrate | 0.0046 |
| | Sodium molybdate dihydrate | 0.0028 |
| | Iron sulfate monohydrate | 0.015 |
| | Citric acid monohydrate | 0.4 |

Generation and Identification of Fusaricidin Improved Mutants of Wildtype Strain LU17007

The tested strains in this experiment were obtained through random mutagenesis of the Paenibacillus polymyxa wildtype strain LU17007 adding the mutagenic agent NTG (N-methyl-N′-nitro-N-nitrosoguanidine) to a thawed cryo vial. The mutagenized culture was plated on LB plates and incubated for 3 days at 28° C. to obtain single colonies. To identify mutants with improved fusaricidin yield, single colonies were picked and then transferred into a 48-microwell plates (0.8 ml) with the preculture medium PX-79 and incubated for 24 hours at 33° C. and 220 rpm at 5 cm shaking diameter. 2% (v/v) of seed culture were used to inoculate a 48-microwell plates with 0.6 ml of the main culture medium PX-143. The cultivation took place for 32 hours at 33° C. and 220 rpm at 50 mm shaking diameter.

Offline samples were taken at the end of the cultivation to measure OD600 and fusaricidin. The OD600 of all culture wells were measured in 48-well microtiter using a microplate reader. For fusaricidin measurement the extraction was by mixing 50 μl broth+950 μl acetonitrile. After centrifugation at 16200 rpm for 10 min the supernatants were transferred to HPLC vials and quantified with the short-HPLC method.

Short PLC Method:
Column: Thermo Hypersil GOLD C18, 100×4.6 mm; 5 μm
Pre-column: Thermo C18 compatible
Temperature: 40° C.
Flowrate: 2.00 ml/min
Injection volume: 1.0 μl
Wash/flush: 20% MeOH in water (2500 μl/5000 μl)
Injector throw: aft 2 min
Detection: UV 200 nm
Separates: FusD (2.2 min) and FusB (3.4 min)
linearity: up to 1 g/l of undiluted analyte at 1 μl injection
Run time: 10 min
Eluent A: H₂O with 0.1% H3PO4
Eluent B: acetonitrile

TABLE
HPLC gradient program

| Time [min] | % A | % B |
|---|---|---|
| 0.00 | 70 | 30 |
| 4.00 | 70 | 30 |
| 4.01 | 5 | 95 |
| 6.00 | 5 | 95 |
| 6.01 | 70 | 30 |
| 10.0 | 70 | 30 |

Media Performance Comparison

Cultivation Conditions

Of the fusaricidin-producing mutants, 18 strains were randomly picked and fermented as follows:

As pre-culture 30 ml of PX-79 medium was inoculated with the respective Paenibacillus polymyxa strains plated on an ISP2 cultivation plate as described in example 10. The cultivation took place for 24 h at 33° C. at the shaking frequency of 150 rpm and 25 mm shaking diameter in a 250 ml baffled shake flask closed with silicon plugs.

The main cultures were then inoculated with 2% (v/v) of the pre-culture. The performance of PX-143 medium was evaluated in comparison to the above minimal cultivation medium. The main culture cultivations were conducted in 250 ml shake flasks with 30 ml culture medium, shaken at 250 rpm at 50 mm shaking diameter for 48 h at 33° C.

Offline samples were taken at the end of a cultivation (48 h) for determination of optical density at 600 nm and fusaricidin concentrations. For the fusaricidin measurement, 50 μl of culture broth was mixed together with 950 μl acetonitrile-water (1:1) mixture for extraction. The sample was treated for 30 min at 20° C. in an ultrasonic bath. The sample then was centrifuged for 5 min at 14000 rpm and the supernatant filtered into a HPLC vial for measurement. Fusaricidin concentrations were measured as described above in this example 13.

FIG. 14 shows the normalized concentration of total fusaricidins A, B and D obtained after the above cultivations for the 18 Paenibacillus polymyxa chemical NTG mutants compared to the wild type progenitor in different media. Fusaricidin production by the mutants was significantly increased in the above minimal medium (labelled “MM” in the figure) according to the present invention compared to the complex medium PX143 in 15 out of 18 mutants tested. Furthermore, for 7 mutants fusaricidin production was clearly higher in the minimal medium compared to fusaricidin production of the wild type strain in the complex medium. Only 4 mutants showed a clearly improved fusaricidin production in the complex medium. Overall, the figure shows that the advantageous effects (in particular increased fusaricidin production) of a minimal medium according to the present invention is not confined to a particular strain but is widely applicable.

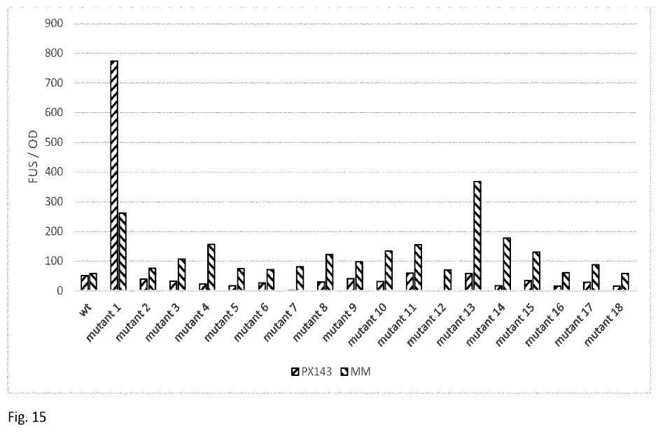

FIG. 15 shows the total fusaricidin A, B and D yield for the same shake flask cultivations as depicted in FIG. 14. Fusaricidin yield is approximated by dividing total fusaricidin A, B and D concentration by OD600 of the respective culture after 48 h of fermentation. For all mutants fusaricidin yield is increased, compared to the wild type, in minimal medium according to the present invention (labelled “MM” in the figure). Only one mutant shows improved fusaricidin yield compared to the wild type when cultivated in complex medium. Again, the figure shows that the benefit (here: improved yield) of a minimal medium of the present invention is widely applicable and not confined to a specific Paenibacillus strain.

1. A fermentation medium for production of a plant health promoting microorganism, comprising
nicotinic acid and biotin,
wherein a concentration of nicotinic acid in the fermentation medium is at least 0.1 mg/l, and
wherein a concentration of biotin in the fermentation medium is at least 0.01 mg/l;
and
methionine,
wherein a concentration of methionine in the fermentation medium is at least 0.01 g/l.

2. The fermentation medium according to claim 1, further comprising a slow-release amino acid source selected from one or more of the group consisting of:
one or more protein sources selected from the group consisting of corn steep liquor, milk protein, skim milk protein, whey protein, casein, pea protein, cotton seed protein, wheat gluten protein, porcine protein, bovine protein, gelatin, egg protein, fish protein, microbial protein, soy protein, and soy meal,
one or more protein hydrolysate sources selected from the group consisting of hydrolysates of one or more of the one or more protein sources, tryptose (peptone from protein mixture, tryptic digest), proteose-peptone, peptone from casein, peptone from gelatin, lactalbumin hydrolysate, liver hydrolysate, peptone from meat, peptone from porcine heart, peptone from plant protein, peptone from broadbean, gluten hydrolysate from maize, peptone from pea, peptone from potatoes, peptone from soybean, peptone from soybean meal, peptone from wheat, peptone from fungal protein, and potato infusion powder, and
one or more discouraged sources selected from the group consisting of brain extract, brain heart infusion, heart extract, heart infusion powder, meat extract, yeast autolysate, and yeast extract,
wherein a total concentration of the slow-release amino acid source in the fermentation medium is 0-100 g/l.

3. The fermentation medium according to claim 2, wherein a total concentration of yeast extract and yeast autolysate in the fermentation medium is 0-8 g/l.

4. The fermentation medium according to claim 1, further comprising a sugar source selected from the group consisting of glucose, dextrose, starch, fructose, galactose, xylose, xylitol, inulin, sorbitol, fucose, molasses, sucrose, lactose, glycerol, pectin, galacturonic acid, maltose, maltodextrin, maltotriose or higher maltooligosacharides or maltose syrup, and mixtures thereof,
wherein a total concentration of the sugar source is at least 5 g/l.

5. The fermentation medium according to claim 1, further comprising
MnSO₄*H₂O having a concentration of 1-1000 mg/l,
SCuSO₄*5H₂O having a concentration of 0.1-100 mg/l,
Na₂MoO₄*2H₂O having a concentration of 0.1-10 mg/l,
Fe₂(SO₄)₃*H₂O having a concentration of 0.8-1000 mg/l,
citric acid having a concentration of 0.1-100 g/l, and
Ca(NO₃)₂*4H₂O having a concentration of 0-3 g/l.

6. The fermentation medium according to claim 1, further comprising
one or more of the following amino acids:
Histidine having a concentration of at least 10 mg/l,
Proline having a concentration of at least 10 mg/l,
Arginine having a concentration of at least 10 mg/l,
Glutamate having a concentration of at least 10 mg/l,
and optionally one or more of the following amino acids
Cysteine having a concentration of at least 10 mg/l, and
Tryptophan having a concentration of at least 10 mg/l.

7. The fermentation medium according to claim 1, wherein
a total concentration of a discouraged slow-release amino acid source in the fermentation medium is 0-3 g/l, and a concentration of yeast extract and yeast autolysate in the fermentation medium is 0-3 g/l,
a total concentration of a sugar source in the fermentation medium is 10-100 g/l, and
a total concentration of a slow-release amino acid protein or protein hydrolysate source is 5-100 g/l.

8. A fermentation method comprising cultivating a microorganism culture comprising one or more plant health promoting microorganisms,
wherein contents of the fermentation medium according to claim 1 is provided to the culture within a period of at most 72 h.

9. The fermentation method according to claim 8, wherein
the microorganism culture comprises one or more biocontrol microorganisms selected from the group of taxonomic ranks consisting of:
phylum Firmicutes;
phylum Proteobacteria, of class Gammaproteobacteria;
phylum Proteobacteria, of class Betaproteobacteria;
phylum Proteobacteria, of class Alphaproteobacteria, of order Rhizobiales;
phylum Proteobacteria, of class Alphaproteobacteria, of order Sphingomonadales
phylum Actinobacteria, of class Actinobacteria, of order Streptomycetales,
phylum Bacteroidetes; and
phylum Actinobacteria, of class Actinobacteria, of order Corynebacteriales;
and wherein the microorganism culture is
a mixed culture consisting of different species of microorganisms and/or different strains of a species of microorganisms, or
a pure culture consisting of one species of microorganisms.

10. The fermentation method according to claim 8, wherein
a) during cultivation, at least one microorganism of the microorganism culture produces spores, and the spores are harvested, and/or
b) a cell free suspension is harvested.

11. A plant health promotion composition, obtainable or obtained by the method according to claim 10,
optionally further comprising a stabilizer, a fusaricidin and/or
a) one or more microbial pesticides with fungicidal, bactericidal, viricidal and/or plant defense activator activity,
b) one or more biochemical pesticides with fungicidal, bactericidal, viricidal and/or plant defense activator activity,
c) one or more microbial pesticides with insecticidal, acaricidal, molluscidal and/or nematicidal activity,
d) one or more biochemical pesticides with insecticidal, acaricidal, molluscidal, pheromone and/or nematicidal activity, and/or
e) one or more fungicides selected from the group consisting of respiration inhibitors, sterol biosynthesis inhibitors, nucleic acid synthesis inhibitors, inhibitors of cell division and cytoskeleton formation or function, inhibitors of amino acid and protein synthesis, signal transduction inhibitors, lipid and membrane synthesis inhibitors, inhibitors with multi-site action, cell wall synthesis inhibitors, plant defense inducers, and fungicides with unknown mode of action.

12. A plant material including a surface and comprising on the surface a composition according to claim 11.

13. A method of using a composition according to claim 11, the method comprising using the composition for preventing, limiting or reducing a phytopathogenic fungal disease and/or for improving the health of a plant and/or for increasing yield of plants.

14. The method according to claim 13, wherein the fungal disease is
i) selected from the group consisting of white blister, downy mildews, powdery mildews, clubroot, Sclerotinia rot, Fusarium wilts and rots, Botrytis rots, anthracnose, Rhizoctonia rots, damping-off, cavity spot, tuber diseases, rusts, black root rot, target spot, Aphanomyces root rot, Ascochyta collar rot, gummy stem blight, Alternaria leaf spot, black leg, ring spot, late blight, Cercospora, leaf blight, Septoria spot, leaf blight, and a combination thereof, and/or
ii) caused or aggravated by a microorganism selected from the group of taxonomic ranks consisting of:
class Sordariomycetes;
class Leotinomycetes;
class Dothideomycetes of order Pleosporales;
class Dothideomycetes of order Pleosporales;
class Dothideomycetes of order Botryosphaeriales;
class Dothideomycetes of order Capnodiales;
class Agraricomycetes;
class Pucciniomycetes;
class Ustilaginomycetes;
class Oomycota; and
class Oomycota of order Peronosporales.

15. A method for preventing, limiting or reducing a phytopathogenic fungal disease and/or for increasing the health of a plant, comprising applying an effective amount of the composition according to claim 11 to the plant, a part or propagation material thereof or to soil where the plant is to grow.

16. The fermentation medium according to claim 1, further comprising a slow-release amino acid source selected from one or more of the group consisting of:
one or more protein sources selected from the group consisting of corn steep liquor, milk protein, skim milk protein, whey protein, casein, pea protein, cotton seed protein, wheat gluten protein, porcine protein, bovine protein, gelatin, egg protein, fish protein, microbial protein, soy protein, and soy meal,
one or more protein hydrolysate sources selected from the group consisting of hydrolysates of one or more of the one or more protein sources, tryptose, proteose-peptone, peptone from casein, peptone from gelatin, lactalbumin hydrolysate, liver hydrolysate, peptone from meat, peptone from porcine heart, peptone from plant protein, peptone from broadbean, gluten hydrolysate from maize, peptone from pea, peptone from potatoes, peptone from soybean, peptone from soybean meal, peptone from wheat, peptone from fungal protein, and potato infusion powder, and
one or more discouraged sources selected from the group consisting of brain extract, brain heart infusion, heart extract, heart infusion powder, meat extract, yeast autolysate, and yeast extract,
wherein a total concentration of the slow-release amino acid source in the fermentation medium is 0-100 g/l.

17. The fermentation medium according to claim 2, wherein a total concentration of yeast extract and yeast autolysate in the fermentation medium is 0-8 g/l, and wherein a total concentration of discouraged sources in the fermentation medium is 0-8 g/l.

18. The fermentation medium according to claim 1, further comprising a sugar source selected from the group consisting of glucose, dextrose, starch, fructose, galactose, xylose, xylitol, inulin, sorbitol, fucose, molasses, sucrose, lactose, glycerol, pectin, galacturonic acid, maltose, maltodextrin, maltotriose or higher maltooligosacharides or maltose syrup, and mixtures thereof, wherein a total concentration of the sugar source is at least 40 g/l.

19. A fermentation medium for production of a plant health promoting microorganism, comprising
nicotinic acid and biotin, wherein a concentration of nicotinic acid in the fermentation medium is at least 2 mg/l, and wherein a concentration of biotin in the fermentation medium is at least 0.01 mg/l; and
methionine, wherein a concentration of methionine in the fermentation medium is at least 0.01 g/l.

20. A fermentation medium for production of a plant health promoting microorganism, comprising
nicotinic acid and biotin, wherein a concentration of nicotinic acid in the fermentation medium is at least 0.1 mg/l, and wherein a concentration of biotin in the fermentation medium is at least 0.05 mg/l; and
methionine, wherein a concentration of methionine in the fermentation medium is at least 0.01 g/l.
